(19) United States
(12) Patent Application Publication
(10) Pub. No.: US 2024/0050506 A1
(43) Pub. Date: Feb. 15, 2024

(54) STEVIOL GLYCOSIDE COMPOSITIONS WITH REDUCED SURFACE TENSION

(71) Applicant: CARGILL, INCORPORATED, Wayzata, MN (US)

(72) Inventors: Dan S. GASPARD, Victoria, MN (US); Adam John STEINBACH, Bloomington, MN (US); Adam T. ZARTH, St. Louis Park, MN (US)

(73) Assignee: CARGILL, INCORPORATED, Wayzata, MN (US)

(21) Appl. No.: 18/328,066

(22) Filed: Jun. 2, 2023

Related U.S. Application Data
(63) Continuation of application No. 16/374,388, filed on Apr. 3, 2019, now Pat. No. 11,701,400.
Application No. 16/374,388 is a continuation of application No. PCT/US2018/054804, filed on Oct. 8, 2018.
(60) Provisional application No. 62/569,279, filed on Oct. 6, 2017.
(60) Provisional application No. 62/676,722, filed on May 25, 2018.

(51) Int. Cl. A61K 36/28; A23L 2/56; A23L 2/60; C07H 15/256; C07K 1/18; C07K 1/16; A23L 27/30; A23L 2/68; A23L 27/00; A23L 5/40; A23F 3/34; A61K 31/192; A61K 31/235; B01D 15/36

(52) U.S. Cl. CPC A61K 36/28; A23L 2/56; A23L 2/60; C07H 15/256; C07K 1/18; C07K 1/16; A23L 27/30; A23L 2/68; A23L 27/36; A23L 27/88; A23L 27/39; A23L 5/40; A23F 3/34; A61K 31/192; A61K 31/235; B01D 15/361; A23V 2002/00; A23V 2200/15; A23V 2250/258; A61K 2236/31; A61K 2236/39

(57) ABSTRACT

A steviol glycoside composition with reduced surface tension. The reduced surface tension steviol glycoside composition includes an aqueous solution of a steviol glycoside and a surface tension reducing compound in an amount effective to reduce surface tension. Method for reducing surface tension in a steviol glycoside solution includes contacting a steviol glycoside and a surface tension reducing compound.

### CROSS REFERENCE TO RELATED APPLICATIONS

This application is a Continuation of U.S. application Ser. No. 16/374,388, filed Apr. 3, 2019, which is a Continuation of International Application No. PCT/US2018/054804, filed Oct. 8, 2018, which claims the benefit of U.S. Provisional Application No. 62/569,279, filed Oct. 6, 2017, each of which is incorporated by reference herein in its entirety. This application claims the benefit of U.S. Provisional Application Ser. No. 62/676,722, filed May 2018, which application is incorporated by reference herein in its entirety.

### FIELD

The present disclosure generally relates to steviol glycoside compositions with reduced surface tension having one or more surface tension reducing compounds and one or more steviol glycosides. The present disclosure also discloses methods of making and using these steviol glycoside compositions with reduced surface tension.

### BACKGROUND

Traditionally, sugars such as sucrose and fructose have been used to provide a sweetened taste to foods, beverages, pharmaceuticals, and oral hygiene/cosmetic products. While these sugars can provide a taste preferred by consumers, they are caloric. In the last decades, as consumers have become more conscious of caloric intake, there has been increased interest in reducing the amount of caloric sugars in products. One approach to reduce the amount of these sugars has been to replace caloric sugars with non-caloric sweeteners. Non-caloric sweeteners can provide a sweetened taste to foods, beverages, pharmaceuticals, and oral hygiene/cosmetic products without adding calories. Steviol glycosides are an example of high intensity non-caloric sweeteners that can provide a sweetened taste to products without adding calories.

Steviol glycosides are glycosides of steviol, a diterpene compound and are about 150 to 450 times sweeter than sugar. Examples of steviol glycosides are described in WO 2013/096420 (see, e.g., listing in FIG. 1); and in Ohta et. al., “Characterization of Novel Steviol Glycosides from Leaves of Stevia rebaudiana Morita,” J. Appl. Glycosi., 57, 199-209 (2010) (See, e.g., Table 4 at p. 204). Structurally, the diterpene glycosides are characterized by a single base, steviol, and differ by the presence of carbohydrate residues at positions C13 and C19, as presented in FIGS. 2a-2k of PCT Patent Publication WO 2013/096420. Steviol glycosides can include one or more of dulcoside A, stevioside, steviolbioside, rubusoside and/or one or more of rebaudioside A, B, C, D, E, F, G, H, I, J, K, L, M, N, and/or O.

While steviol glycoside can provide a sweetened taste to products, there can be challenges to preparing products with steviol glycoside. In some cases, there may be advantages to preparing a product comprising steviol glycoside composition with reduced surface tension. For example, consumers may prefer a product comprising a steviol glycoside composition with reduced surface tension. Such products can include beverages such as carbonated soda drinks, flavored waters, carbonated flavored waters, and other beverages. Such products can also include dry sweetener compositions, dry drink mixes, and concentrated liquid drink mixes.

Although conventional surfactants can be used to reduce surface tension in food applications, there may be drawbacks to their use with steviol glycoside products. For example, many consumers select steviol glycoside products because these consumers prefer products with natural ingredients. These consumers may not desire a steviol glycoside product with conventional surfactant agents. Likewise, some consumers may prefer a steviol glycoside product made from natural ingredients. Similarly, some consumers may prefer a steviol glycoside product with an ingredient label that lists natural ingredients. Therefore, these consumers may prefer steviol glycoside products with a surface tension reducing compound isolated from a natural source such as a botanical source.

It is an object of the present disclosure to provide a surface tension reducing compound for steviol glycoside compositions with reduced surface tension, for example in the preparation of foods, beverages, pharmaceuticals, and oral hygiene/cosmetic products with steviol glycoside. It is also an object of the present disclosure to provide a surface tension reducing compound isolated from a natural source such as a botanical source.

### SUMMARY

The present disclosure generally relates to steviol glycoside compositions comprising a steviol glycoside and a surface tension reducing compound in an amount effect to reduce surface tension. One aspect provides a steviol glycoside composition with reduced surface tension, the composition comprising an aqueous solution of a steviol glycoside and a surface tension reducing compound in an amount effective to reduce surface tension by at least 10% when compared to a steviol glycoside solution without a surface tension reducing compound. In some aspects, the steviol glycoside composition with reduced surface tension comprises the surface tension reducing compound in an amount effective to reduce surface tension by at least 20%, at least 30%, at least 40%, at least 50%, at least 60%, at least 70%, at least 80% or at least 90% compared to a steviol glycoside solution without the surface tension reducing compound. In some aspects, the steviol glycoside comprises one or more steviol glycosides selected from the group consisting of rebaudioside A, rebaudioside B, rebaudioside C, rebaudioside D, rebaudioside E, rebaudioside F, rebaudioside M, rubusoside, dulcoside A, rebaudioside I, rebaudioside Q, rebaudioside N, rebaudioside O, 1,2-stevioside, 1,3-stevioside, steviol-1,2-bioside, steviol-1,3-bioside, steviol-13-O-glucoside (13-SMG), and steviol-19-O-glucoside (19-SMG). In other aspects, the steviol glycoside comprises one or more steviol glycosides selected from the group consisting of rebaudioside D and rebaudioside M.

In some aspects, the surface tension reducing compound comprises one or more compounds selected from the group consisting of a quinic acid, caffeic acid, ferulic acid, sinapic acid, p-coumaric acid, an ester of quinic acid, an ester of caffeic acid, an ester of ferulic acid, an ester of sinapic acid, an ester of p-coumaric acid, an ester of caffeic acid and quinic acid, an ester of caffeic acid and quinic acid comprising a single caffeic acid moiety, an ester of caffeic acid and quinic acid comprising more than one caffeic acid moiety, an ester of ferulic acid and quinic acid, an ester of ferulic acid and quinic acid comprising a single ferulic acid moiety, an ester of ferulic acid and quinic acid comprising more than one ferulic acid moiety, an ester of sinapic acid and quinic acid, an ester of sinapic acid and quinic acid comprising a single sinapic acid moiety, an ester of sinapic acid and quinic acid comprising more than one sinapic acid moiety, an ester of p-coumaric acid and quinic acid, an ester of p-coumaric acid and quinic acid comprising a single p-coumaric acid moiety, an ester of p-coumaric acid and quinic acid comprising more than one p-coumaric acid moiety, a caffeic ester of 3-(3,4-dihydroxyphenyl)lactic acid, an ester of caffeic acid and tartaric acid, an ester of caffeic acid and tartaric acid comprising a single caffeic acid moiety, an ester of caffeic acid and tartaric acid comprising more than one caffeic acid moiety, salt thereof and/or isomers thereof. In other aspects, the surface tension reducing compound comprises one or more compounds selected from the group consisting of chlorogenic acid, neochlorogenic acid, cryptochlorogenic acid, 3-O-caffeoylquinic acid, 4-O-caffeoylquinic acid, 5-O-caffeoylquinic acid, 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, 4,5-dicaffeoylquinic acid, 3-O-feruloylquinic acid, 4-O-feruloylquinic acid, 5-O-feruloylquinic acid, 3,4-diferuloylquinic acid, 3,5-diferuloylquinic acid, 4,5-diferuloylquinic acid, rosmarinic acid, cichoric acid, caftaric acid, monocaffeoyltartaric acid, dicaffeoyltartaric acid and salts and/or isomers thereof. In some aspects, the surface tension reducing compound comprises one or more compounds selected from the group consisting of chlorogenic acid, neochlorogenic acid, cryptochlorogenic acid, 3-O-caffeoylquinic acid, 4-O-caffeoylquinic acid, 5-O-caffeoylquinic acid, 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid. In other aspects, the surface tension reducing compound is enriched for one or more dicaffeoylquinic acids, and salts thereof. In some aspects, the surface tension reducing compound comprises 10% or more, 15% or more, 20% or more, 25% or more, 30% or more, 35% or more, 40% or more, 45% or more, or 50% or more dicaffeoylquinic acids, and salts thereof.

In some aspects, the composition comprises a final concentration of steviol glycoside of between about 1 ppm to about 1000 ppm, about 1 ppm to about 10000 ppm, about 1 ppm to about 100000 ppm, about 1 ppm to about 200000 ppm, or about 1 ppm to about 300000 ppm. In some aspects, the composition comprises a final concentration of steviol glycoside of between about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In other aspects, the composition comprises a final concentration of surface tension reducing compound of between about 1 ppm to about 1000 ppm, about 1 ppm to about 10000 ppm, about 1 ppm to about 100000 ppm, about 1 ppm to about 200000 ppm, or about 1 ppm to about 300000 ppm. In some aspects, the composition comprises a final concentration of surface tension reducing compound of between about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, the composition comprises a ratio of surface tension reducing compound to steviol glycoside of 0.1:1 to 5:1, about 0.5:1 to 4:1, or about 1:1 to 3:1.

In some aspects, the surface tension reducing compound is prepared from a botanical source. In some aspects, the botanical source is selected from the group consisting of eucommoia ulmoides, honeysuckle, nicotiana benthamiana, globe artichoke, cardoon, Stevia, Stevia rebaudiana, monkfruit, coffee, coffee beans, green coffee beans, tea, white tea, yellow tea, green tea, oolong tea, black tea, red tea, post-fermented tea, bamboo, heather, sunflower, blueberries, cranberries, bilberries, grouseberries, whortleberry, lingonberry, cowberry, huckleberry, grapes, chicory, eastern purple coneflower, echinacea, Eastern pellitory-of-the-wall, Upright pellitory, Lichwort, Greater celandine, Tetterwort, Nipplewort, Swallowwort, Bloodroot, Common nettle, Stinging nettle, Potato, Potato leaves, Eggplant, Aubergine, Tomato, Cherry tomato, Bitter apple, Thorn apple, Sweet potato, apple, Peach, Nectarine, Cherry, Sour cherry, Wild cherry, Apricot, Almond, Plum, Prune, Holly, Yerba mate, Mate, Ilex paraguariensis, Guayusa, Yaupon Holly, Kuding, Guarana, Cocoa, Cocoa bean, Cacao, Cacao bean, Kola nut, Kola tree, Cola nut, Cola tree, Hornwort, Ostrich fern, Oriental ostrich fern, Fiddlehead fern, Shuttlecock fern, Oriental ostrich fern, Asian royal fern, Royal fern, Bracken, Brake, Common bracken, Eagle fern, Eastern brakenfern, dandelion, algae, seagrasses, Clove, Cinnamon, Indian bay leaf, Nutmeg, Bay laurel, Bay leaf, Basil, Great basil, Saint-Joseph's-wort, Thyme, Sage, Garden sage, Common sage, Culinary sage, Rosemary, Oregano, Wild marjoram, Marjoram, Sweet marjoram, Knotted marjoram, Pot marjoram, Dill, Anise, Star anise, Fennel, Florence fennel, Tarragon, Estragon, Mugwort, Licorice, Liquorice, Soy, Soybean, Soyabean, Soya vean, Wheat, Common wheat, Rice, Canola, Broccoli, Cauliflower, Cabbage, Bok choy, Kale, Collard greens, Brussels sprouts, Kohlrabi, Winter's bark, Elderflower, Assa-Peixe, Greater burdock, Valerian, and Chamomile. In some aspects, the botanical source is yerba mate, chicory, rosemary, and/or Stevia.

One aspect provides a beverage concentrate solution with reduced surface tension, the solution comprising a steviol glycoside and a surface tension reducing compound in an amount effective to reduce surface tension by at least 10% during at least six-fold dilution of the concentrate solution. In some aspects, the steviol glycoside comprises one or more steviol glycosides selected from the group consisting of rebaudioside D and rebaudioside M. In some aspects, the surface tension reducing compound comprises one or more compounds selected from the group consisting of a quinic acid, caffeic acid, ferulic acid, sinapic acid, p-coumaric acid, an ester of quinic acid, an ester of caffeic acid, an ester of ferulic acid, an ester of sinapic acid, an ester of p-coumaric acid, an ester of caffeic acid and quinic acid, an ester of caffeic acid and quinic acid comprising a single caffeic acid moiety, an ester of caffeic acid and quinic acid comprising more than one caffeic acid moiety, an ester of ferulic acid and quinic acid, an ester of ferulic acid and quinic acid comprising a single ferulic acid moiety, an ester of ferulic acid and quinic acid comprising more than one ferulic acid moiety, an ester of sinapic acid and quinic acid, an ester of sinapic acid and quinic acid comprising a single sinapic acid moiety, an ester of sinapic acid and quinic acid comprising more than one sinapic acid moiety, an ester of p-coumaric acid and quinic acid, an ester of p-coumaric acid and quinic acid comprising a single p-coumaric acid moiety, an ester of p-coumaric acid and quinic acid comprising more than one p-coumaric acid moiety, a caffeic ester of 3-(3,4-dihydroxyphenyl)lactic acid, an ester of caffeic acid and tartaric acid, an ester of caffeic acid and tartaric acid comprising a single caffeic acid moiety, an ester of caffeic acid and tartaric acid comprising more than one caffeic acid moiety, salt thereof and/or isomers thereof. In some aspects, the surface tension reducing compound comprises one or more compounds selected from the group consisting of chlorogenic acid, neochlorogenic acid, cryptochlorogenic acid, 3-O-caffeoylquinic acid, 4-O-caffeoylquinic acid, 5-O-caffeoylquinic acid, 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, 4,5-dicaffeoylquinic acid, 3-O-feruloylquinic acid, 4-O-feruloylquinic acid, 5-O-feruloylquinic acid, 3,4-diferuloylquinic acid, 3,5-diferuloylquinic acid, 4,5-diferuloylquinic acid, rosmarinic acid, cichoric acid, caftaric acid, monocaffeoyltartaric acid, dicaffeoyltartaric acid and salts and/or isomers thereof. In some aspects, the surface tension reducing compound comprises one or more compounds selected from the group consisting of chlorogenic acid, neochlorogenic acid, cryptochlorogenic acid, 3-O-caffeoylquinic acid, 4-O-caffeoylquinic acid, 5-O-caffeoylquinic acid, 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid. In some aspects, the surface tension reducing compound is enriched for one or more dicaffeoylquinic acids, and salts thereof. In some aspects, the surface tension reducing compound comprises 10% or more, 15% or more, 20% or more, 25% or more, 30% or more, 35% or more, 40% or more, 45% or more, or 50% or more dicaffeoylquinic acids, and salts thereof.

In some aspects, the concentrate solution further comprises a steviol glycoside concentration of between about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, concentrate solution further comprises a concentration of surface tension reducing compound of between about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, the concentrate solution further comprises a concentration of surface tension reducing compound of between about 1000 ppm to about 10000 ppm, about 1000 ppm to about 9000 ppm, 1000 ppm to about 8000 ppm, 1000 ppm to about 7000 ppm, 1000 ppm to about 6000 ppm, 1000 ppm to about 5000 ppm, or 1800 ppm to about 5400 ppm. In some aspects, the concentrate solution further comprises a ratio of surface tension reducing compound to steviol glycoside of 0.1:1 to 5:1, about 0.5:1 to 4:1, or about 1:1 to 3:1.

In some aspects, the concentrate solution further comprises surface tension reducing compound prepared from yerba mate, chicory, rosemary, and/or Stevia. In some aspects, the concentrate solution has a pH of 1.5 to 4.

One aspect provides a steviol glycoside composition comprising an admixture of steviol glycoside and surface tension reducing compound, wherein an aqueous solution prepared from the steviol glycoside composition has a reduced surface tension compared to a steviol glycoside solution without surface tension reducing compound. In some aspects, the steviol glycoside comprises one or more steviol glycosides selected from the group consisting of rebaudioside D and rebaudioside M. In some aspects, the steviol glycoside comprises rebaudioside A. In some aspects, the surface tension reducing compound comprises one or more compounds selected from the group consisting of chlorogenic acid, neochlorogenic acid, cryptochlorogenic acid, 3-O-caffeoylquinic acid, 4-O-caffeoylquinic acid, 5-O-caffeoylquinic acid, 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, 4,5-dicaffeoylquinic acid, 3-O-feruloylquinic acid, 4-O-feruloylquinic acid, 5-O-feruloylquinic acid, 3,4-diferuloylquinic acid, 3,5-diferuloylquinic acid, 4,5-diferuloylquinic acid, rosmarinic acid, cichoric acid, caftaric acid, monocaffeoyltartaric acid, dicaffeoyltartaric acid and salts and/or isomers thereof. In some aspects, the surface tension reducing compound comprises one or more compounds selected from the group consisting of chlorogenic acid, neochlorogenic acid, cryptochlorogenic acid, 3-O-caffeoylquinic acid, 4-O-caffeoylquinic acid, 5-O-caffeoylquinic acid, 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid. In some aspects, the surface tension reducing compound is enriched for one or more dicaffeoylquinic acids, and salts thereof. In some aspects, the surface tension reducing compound comprises 10% or more, 15% or more, 20% or more, 25% or more, 30% or more, 35% or more, 40% or more, 45% or more, or 50% or more dicaffeoylquinic acids, and salts thereof. In some aspects, the admixture comprises a ratio of surface tension reducing compound to steviol glycoside of 0.1:1 to 5:1, about 0.5:1 to 4:1, or about 1:1 to 3:1. In some aspects, the surface tension reducing compound is prepared from one or more botanical sources selected from the group consisting of yerba mate, chicory, rosemary, and Stevia. In some aspects, the steviol glycoside composition further comprises a buffering system to provide a pH of 1.5 to 4 in to the aqueous solution prepared from the steviol glycoside composition.

One aspect provides a method for reducing surface tension in a steviol glycoside solution, the method comprising contacting a steviol glycoside and a surface tension reducing compound to prepare any of the steviol glycoside compositions with reduced surface tension described above. In some aspects, the surface tension is reduced by at least 10%, at least 20%, at least 30%, at least 40%, at least 50%, at least 60%, at least 70%, at least 80% or at least 90% compared to a steviol glycoside solution without the surface tension reducing compound.

One aspect provides a method for producing a reduced surface tension steviol glycoside solution, the method comprising preparing any of the reduced steviol glycoside compositions described above. In some aspects, the surface tension is reduced by at least 10%, at least 20%, at least 30%, at least 40%, at least 50%, at least 60%, at least 70%, at least 80% or at least 90% compared to a steviol glycoside solution without surface tension reducing compound.

### BRIEF DESCRIPTION OF THE FIGURES

FIG. 1 shows surface tension measurements for a control solution of a steviol glycoside blend (RM80), a control solution of surface tension reducing compound(isolated from yerba mate), a test solution of steviol glycoside blend with surface tension reducing compound(isolated from yerba mate), and a test solution of steviol glycoside with chlorogenic acid(commercial preparation).

FIG. 2A shows a kinetic surface plot of surface tension over time for a control solution of a steviol glycoside blend(RM80).

FIG. 2B shows a kinetic surface plot of surface tension over time for a test solution of a steviol glycoside blend(RM80) and a surface tension reducing compound(isolated from yerba mate).

FIG. 3 shows surface tension measurements for solutions of rebaudioside A, rebaudioside D, rebaudioside M, and a blend of rebaudioside A (RA80) with and without surface tension reducing compound(isolated from yerba mate).

FIG. 4 shows surface tension measurements for solutions of rebaudioside M (RM80) with and without surface tension reducing compound (rosmarinic acid or cichoric acid).

FIG. 5 shows a kinetic surface plot of surface tension over time for a control solution of rosmarinic acid and a test solution of steviol glycoside(RM80) and rosmarinic acid.

FIG. 6 shows a kinetic surface plot of surface tension over time for a control solution of cichoric acid and a test solution of steviol glycoside(RM80) and cichoric acid.

### DETAILED DESCRIPTION

The disclosure relates generally to steviol glycoside compositions with reduced surface tension comprising a steviol glycoside and a surface tension reducing compound in an amount effective to reduce surface tension of the steviol glycoside composition.

In some aspects, the term surface tension refers to a tension of a surface film of a liquid caused by attraction of molecules in the surface layer by a bulk of the liquid which tends to minimize surface area. In other aspects, an aqueous solution with reduced surface tension may exhibit an increase in foam formation upon introduction of a gas, may result in a foam that lasts longer over time, and/or may exhibit a smaller bubble size of the gas in the aqueous solution and/or in the foam. For example, a steviol glycoside composition with a surface tension reducing compound can comprise a reduced surface tension when compared with a similar composition without the surface tension reducing compound.

An example of a steviol glycoside composition with reduced surface tension comprises an aqueous solution of a steviol glycoside and surface tension reducing compound in an amount effective to reduce surface tension. In some aspects, a steviol glycoside composition with reduced surface tension comprises an aqueous solution of a steviol glycoside and a surface tension reducing compound in an amount effective to reduce surface tension by at least 10%. In other aspects, a steviol glycoside composition with reduced surface tension comprises an aqueous solution of a steviol glycoside and a surface tension reducing compound in an amount effective to reduce surface tension by at least 10%, 15%, 20%, 25%, 30%, 35%, 40%, 45%, 50%, 55%, 60%, 65%, 70%, 75%, 80%, 85%, 90%, 95% or more.

In some aspects, the aqueous solution comprises primarily water. The aqueous solution can also be buffered with any suitable buffering system, including, but not limited to, one or more buffers such as a phosphate, a citrate, ascorbate, lactate, acetate, and the like. The buffer can comprise 1-1000 mM of the anion component. In other aspects, the aqueous solution comprises a citrate/phosphate buffer. In some aspects, citrate/phosphate buffer can have a pH of 2 to 4.

The amounts of steviol glycoside in the steviol glycoside composition and the surface tension reducing composition can be expressed in relation to one another, or to the total amount of steviol glycosides (TSG), such as a weight percentage of the total amount of steviol glycosides (TSG), or to the total amount of surface tension reducing compound, or a ratio, or range of ratios, expressed as a weight percent, or molar percent.

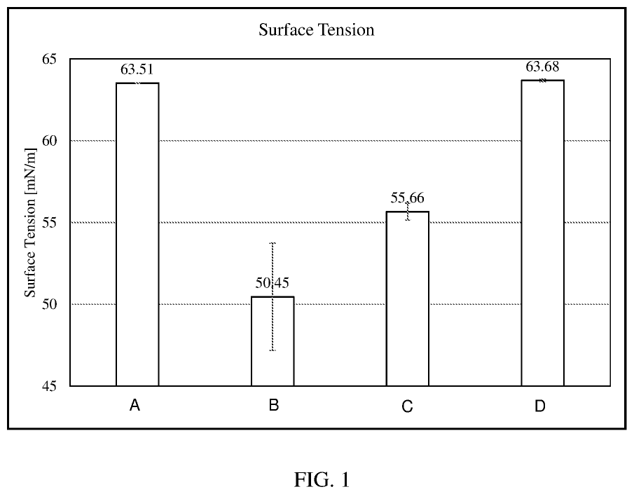

The steviol glycoside composition can include one or more steviol glycosides. In some aspects, the term steviol glycoside refers to Rebaudioside A (Reb A) (CAS #58543-16-1), Rebaudioside B (Reb B) (CAS #58543-17-2), Rebaudioside C (Reb C) (CAS #63550-99-2), Rebaudioside D (Reb D) (CAS #63279-13-0), Rebaudioside E (Reb E) (CAS #63279-14-1), Rebaudioside F (Reb F) (CAS #438045-89-7), Rebaudioside M (Reb M) (CAS #1220616-44-3), Rubusoside (CAS #63849-39-4), Dulcoside A (CAS #64432-06-0), Rebaudioside I (Reb I) (MassBank Record: FU000332), Rebaudioside Q (Reb Q), Rebaudioside O (Reb O), Rebaudioside N (Reb N) (CAS #1220616-46-5), 1,2-Stevioside (CAS # 57817-89-7), 1,3-Stevioside (Reb G), Steviol-1,2-Bioside (MassBank Record: FU000299), Steviol-1,3-Bioside, Steviol-13-O-glucoside (13-SMG), Steviol-19-O-glucoside (19-SMG), and steviol glycoside having 1, 2, 3, 4, 5, 6, 7, 8, 9, 10 or sugar additions (e.g., glucose, rhamnose, and/or xylose), and isomers thereof. See FIG. 1; see also, Steviol Glycosides Chemical and Technical Assessment 69th JECFA, 2007, prepared by Harriet Wallin, Food Agric. Org.

Exemplary steviol glycosides can include rebaudioside M, rebaudioside D, rebaudioside A, rebaudioside B, rebaudioside N, and/or stevioside. In some aspects, one or more of the steviol glycosides are produced by fermentation by an engineered microorganism. For example, rebaudioside D and M can be produced by an engineered organism and then isolated to produce a steviol glycoside composition of primarily rebaudioside D and rebaudioside M as the predominant steviol glycoside species. In other aspects, the steviol glycoside composition can comprise rebaudioside D and rebaudioside M in an amount greater than other steviol glycosides. In some aspects, one or more of the steviol glycosides are isolated from Stevia rebaudiana.

In some aspects, steviol glycosides can include rebaudioside A. For example, steviol glycosides can include blends of rebaudioside A and one or more other steviol glycosides. An example of a blend of rebaudioside A with one or more other steviol glycosides comprises 60% or more rebaudioside A (RA60). Another example of a blend of rebaudioside A with one or more other steviol glycosides comprises 80% or more rebaudioside A (RA80). Another example of a blend of rebaudioside A with one or more other steviol glycosides comprises 95% or more rebaudioside A (RA95). In other aspects, steviol glycosides can include blends of one or more of RebA, RebB, RebC, RebD, RebE, RebF, RebM, rubusoside, dulcoside A, RebI, RebQ, 1,2 stevioside, 1,3 stevioside, steviol-1,2-bioside, steviol-1,3-bioside, 13-SMG, 19-SMG, a tri-glycosylated steviol glycoside, a tetra-glycosylated steviol glycoside, a penta-glycosylated steviol glycoside, a hexa-glycosylated steviol glycoside, a hepta-glycosylated steviol glycoside, and isomers thereof.

In some aspects, the steviol glycoside composition can optionally be described in terms of amounts of rebaudioside M and rebaudioside D. For example, rebaudioside M and rebaudioside D can be present in the composition in a total amount of about 80% (wt) or greater (RM80), 90% (wt) or greater (RM90), or 95% (wt) or greater (RM95), of a total amount steviol glycosides in the composition. Rebaudioside M can be the predominant steviol glycoside in the composition, and can be present, for example, in an amount in the range of about 50% to about 95%, about 70% to about 90%, or about 75% to about 85% of the total amount steviol glycosides in the composition. Rebaudioside D can be in an amount less than Rebaudioside M, such as in an amount in the range of about 5% to about 25%, about 10% to about 20%, or about 10% to about 15% of the total amount steviol glycosides in the composition.

The steviol glycoside composition can optionally be expressed in terms of amounts of other known steviol glycosides that are present in lower amounts. For example, the composition can include one or more of rebaudioside A, rebaudioside B, or stevioside in an amount of about 5% (wt) or less, about 2% (wt) or less, or about 1% (wt) or less, of a total amount of steviol glycosides in the composition.

The amount of steviol glycosides in the steviol glycoside composition with reduced surface tension can vary. Steviol glycosides can be present in the steviol glycoside composition with reduced surface tension in any amount desired for the particular use. For example, steviol glycosides can be present in the steviol glycoside composition with reduced surface tension at a total steviol glycoside concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, steviol glycosides can be present in the steviol glycoside composition with reduced surface tension at a total steviol glycoside concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, steviol glycosides can be present in the steviol glycoside composition with reduced surface tension at a total steviol glycoside concentration from about 1000 ppm to about 5000 ppm, about 2000 ppm to about 5000 ppm, about 3000 ppm to about 5000 ppm, or about 4000 ppm to about 5000 ppm. In some aspects, steviol glycosides can be present in the steviol glycoside composition with reduced surface tension at a total steviol glycoside concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, steviol glycosides can be present in the steviol glycoside composition with reduced surface tension at a total steviol glycoside concentration of or greater than about 200000 ppm. In some aspects, steviol glycosides can be present in the steviol glycoside composition with reduced surface tension at a total steviol glycoside concentration of or greater than about 300000 ppm. Unless otherwise expressly stated, ppm is on a by weight basis.

The amount of an individual steviol glycoside in the steviol glycoside composition with reduced surface tension can vary. For example, an individual steviol glycoside can be present in the steviol glycoside composition with reduced surface tension at a concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, an individual steviol glycoside can be present in the steviol glycoside composition with reduced surface tension at a concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, an individual steviol glycoside can be present in the steviol glycoside composition with reduced surface tension at a concentration from about 1000 ppm to about 5000 ppm, about 2000 ppm to about 5000 ppm, about 3000 ppm to about 5000 ppm, or about 4000 ppm to about 5000 ppm. In some aspects, an individual steviol glycoside can be present in the steviol glycoside composition with reduced surface tension at a concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, an individual steviol glycoside can be present in the steviol glycoside composition with reduced surface tension at a concentration of or greater than about 200000 ppm. In some aspects, an individual steviol glycoside can be present in the steviol glycoside composition with reduced surface tension at a concentration of or greater than about 300000 ppm. Unless otherwise expressly stated, ppm is on a by weight basis

The amount of an individual steviol glycoside in the steviol glycoside composition with reduced surface tension can vary. For example, RebA can be present in the steviol glycoside composition with reduced surface tension at a concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, RebA can be present in the steviol glycoside composition with reduced surface tension at a concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, RebA can be present in the steviol glycoside composition with reduced surface tension at a concentration from about 1000 ppm to about 5000 ppm, about 2000 ppm to about 5000 ppm, about 3000 ppm to about 5000 ppm, or about 4000 ppm to about 5000 ppm. In some aspects, RebA can be present in the steviol glycoside composition with reduced surface tension at a concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, RebA can be present in the steviol glycoside composition with reduced surface tension at a concentration of or greater than about 200000 ppm. In some aspects, RebA can be present in the steviol glycoside composition with reduced surface tension at a concentration of or greater than about 300000 ppm. Unless otherwise expressly stated, ppm is on a by weight basis.

The amount of an individual steviol glycoside in the steviol glycoside composition with reduced surface tension can vary. For example, RebM can be present in the steviol glycoside composition with reduced surface tension at a concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, RebM can be present in the steviol glycoside composition with reduced surface tension at a concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, RebM can be present in the steviol glycoside composition with reduced surface tension at a concentration from about 1000 ppm to about 5000 ppm, about 2000 ppm to about 5000 ppm, about 3000 ppm to about 5000 ppm, or about 4000 ppm to about 5000 ppm. In some aspects, RebM can be present in the steviol glycoside composition with reduced surface tension at a concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, RebM can be present in the steviol glycoside composition with reduced surface tension at a concentration of or greater than about 200000 ppm. In some aspects, RebM can be present in the steviol glycoside composition with reduced surface tension at a concentration of or greater than about 300000 ppm. Unless otherwise expressly stated, ppm is on a by weight basis.

In some aspects, the steviol glycosides comprise a high concentration steviol glycoside solution. In some aspects, a high concentration steviol glycoside solution comprises a steviol glycoside solution comprising a total amount of steviol glycosides (TSG) of 1% or more. A high concentration steviol glycoside solution can comprise a steviol glycoside solution comprising a total amount of steviol glycosides (TSG) of 1%, 2%, 3%, 4%, 5%, 6%, 7%, 8%, 9%, 10% or more. A high concentration steviol glycoside solution can comprise a steviol glycoside solution comprising a total amount of steviol glycosides (TSG) of 11%, 12%, 13%, 14%, 15%, 16%, 17%, 18%, 19%, 20% or more. A high concentration steviol glycoside solution can comprise a steviol glycoside solution comprising a total amount of steviol glycosides (TSG) of 21%, 22%, 23%, 24%, 25%, 26%, 27%, 28%, 29%, 30% or more. A high concentration steviol glycoside solution can comprise one or more of the steviol glycosides described above. For example a high steviol glycoside solution can comprise one or more of RebA, RebB, RebC, RebD, RebE, RebF, RebM, rubusoside, dulcoside A, RebI, RebQ, 1,2 stevioside, 1,3 stevioside, steviol-1,2-bioside, steviol-1,3-bioside, 13-SMG, 19-SMG, a tri-glycosylated steviol glycoside, a tetra-glycosylated steviol glycoside, a penta-glycosylated steviol glycoside, a hexa-glycosylated steviol glycoside, a hepta-glycosylated steviol glycoside, and isomers thereof.

Examples of surface tension reducing compounds include: caffeic acid, an ester of caffeic acid, an ester of caffeic acid and quinic acid, an ester of caffeic acid and quinic acid comprising a single caffeic acid moiety (e.g., chlorogenic acid, cryptochlorogenic acid, and neochlorogenic acid; structures of each are provided herein), an ester of caffeic acid and quinic acid comprising more than one caffeic acid moiety (e.g., 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid; structures of each are provided herein); ferulic acid, an ester of ferulic acid, an ester of ferulic acid and quinic acid, an ester of ferulic acid and quinic acid comprising a single ferulic acid moiety, an ester of ferulic acid and quinic acid comprising more than one ferulic acid moiety; quinic acid, an ester of quinic acid; tartaric acid, a tartaric acid derivative, an ester of tartaric acid (e.g. caftaric acid or cichoric acid), an ester of a tartaric acid derivative, 3-(3,4-dihydroxyphenyl)lactic acid, a 3-(3,4-dihydroxyphenyl)lactic acid derivative, an ester of 3-(3,4-dihydroxyphenyl)lactic acid (e.g. rosmarinic acid), an ester of a 3-(3,4-dihydroxyphenyl)lactic acid derivative, p-coumaric acid, an ester of p-coumaric acid, an ester of p-coumaric acid and quinic acid, an ester of p-coumaric acid and quinic acid comprising a single p-coumaric acid moiety, an ester of p-coumaric acid and quinic acid comprising more than one p-coumaric acid moiety; sinapic acid, an ester of sinapic acid, an ester of sinapic acid and quinic acid, an ester of sinapic acid and quinic acid comprising a single sinapic acid moiety, an ester of sinapic acid and quinic acid comprising more than one sinapic acid moiety; and 3-O-feruloylquinic acid, 4-O-feruloylquinic acid, 5-O-feruloylquinic acid, 3,4-diferuloylquinic acid, 3,5-diferuloylquinic acid, and 4,5-diferuloylquinic acid.

Caffeic acid has the structure:

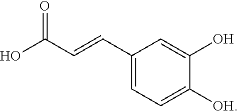 [SMILES: O=C(O)/C=C/c1ccc(O)c(O)c1]

Ferulic acid has the structure:

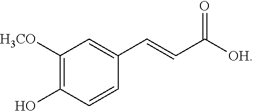 [SMILES: COc1cc(/C=C/C(=O)O)ccc1O]

p-Coumaric acid has the structure:

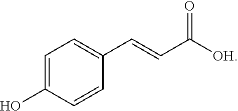 [SMILES: O=C(O)/C=C/c1ccc(O)cc1]

Sinapic acid has the structure:

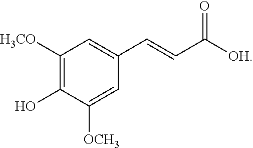 [SMILES: COc1cc(/C=C/C(=O)O)cc(OC)c1O]

Quinic acid has the structure:

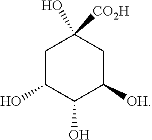 [SMILES: O=C(O)[C@]1(O)C[C@@H](O)[C@@H](O)[C@H](O)C1]

3-(3,4-dihydroxyphenyl)lactic acid has the structure:

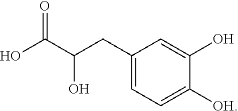 [SMILES: O=C(O)C(O)Cc1ccc(O)c(O)c1]

Tartaric acid has the structure:

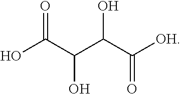

and can be in the D and L forms.

Examples of the esters of the various acids contemplated herein include the ester of caffeic acid and quinic acid, which includes monocaffeoylquinic acids (e.g., chlorogenic acid, neochlorogenic acid, and cryptochlorogenic acid), and dicaffeoylquinic acids (e.g., 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid), and salts thereof:

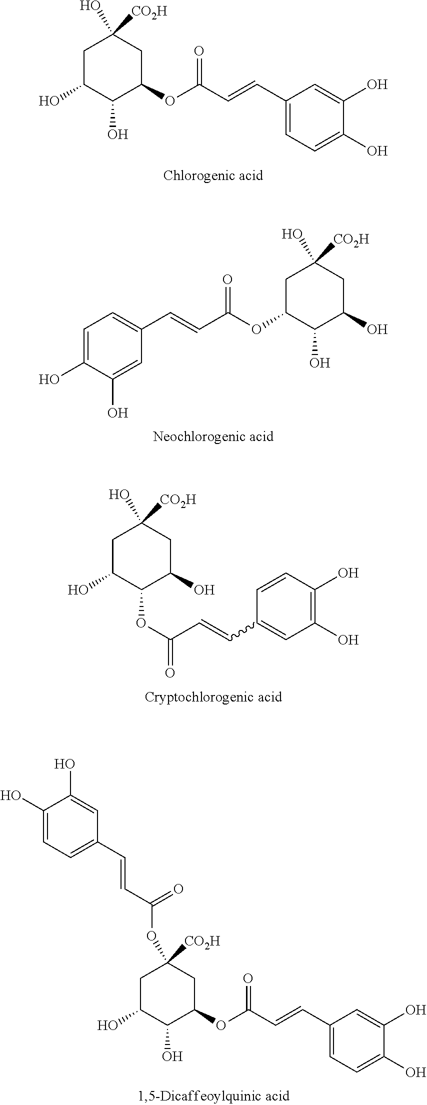 [SMILES: O=C(/C=C/c1ccc(O)c(O)c1)O[C@@H]1C[C@](O)(C(=O)O)C[C@@H](O)[C@H]1O ; O=C(/C=C/c1ccc(O)c(O)c1)O[C@@H]1C[C@@](O)(C(=O)O)C[C@@H](O)[C@@H]1O ; O=C(C=Cc1ccc(O)c(O)c1)O[C@H]1[C@H](O)C[C@](O)(C(=O)O)C[C@H]1O ; O=C(/C=C/c1ccc(O)c(O)c1)O[C@@H]1C[C@](OC(=O)/C=C/c2ccc(O)c(O)c2)(C(=O)O)C[C@@H](O)[C@H]1O]

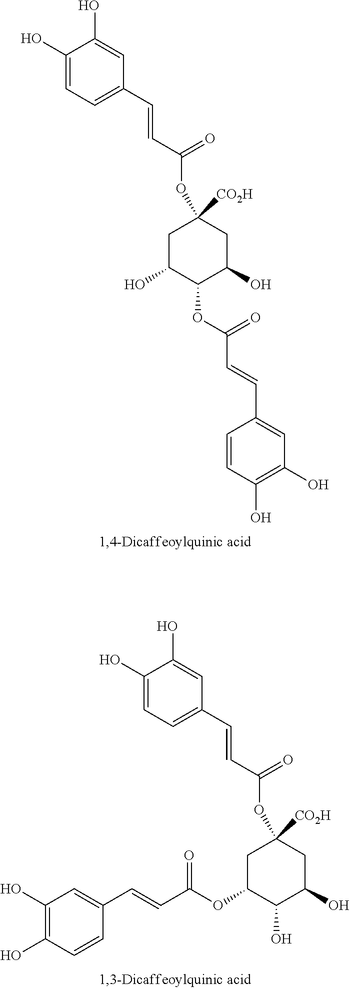 [SMILES: O=C(/C=C/c1ccc(O)c(O)c1)O[C@H]1[C@H](O)C[C@](OC(=O)/C=C/c2ccc(O)c(O)c2)(C(=O)O)C[C@H]1O ; O=C(/C=C/c1ccc(O)c(O)c1)O[C@@H]1C[C@@](OC(=O)/C=C/c2ccc(O)c(O)c2)(C(=O)O)C[C@@H](O)[C@@H]1O]

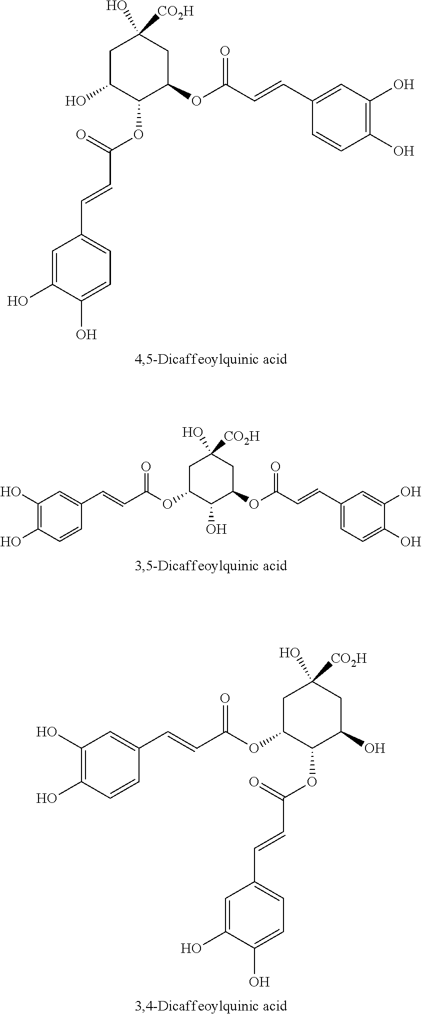 [SMILES: O=C(/C=C/c1ccc(O)c(O)c1)O[C@@H]1[C@H](O)C[C@@](O)(C(=O)O)C[C@H]1OC(=O)/C=C/c1ccc(O)c(O)c1 ; O=C(/C=C/c1ccc(O)c(O)c1)O[C@@H]1C[C@](O)(C(=O)O)C[C@@H](OC(=O)/C=C/c2ccc(O)c(O)c2)[C@H]1O ; O=C(/C=C/c1ccc(O)c(O)c1)O[C@H]1[C@H](O)C[C@](O)(C(=O)O)C[C@H]1OC(=O)/C=C/c1ccc(O)c(O)c1]

with 4,5-dicaffeoylquinic acid, 3,5-dicaffeoylquinic acid, and 3,4-dicaffeoylquinic acid being most prevalent in the compositions contemplated herein and most prevalent in abundant in Stevia, yerba mate, globe artichoke, and green coffee bean.

Examples of the esters of the various acids contemplated herein include the ester of caffeic acid and tartaric acid, which includes cichoric acid having the structure:

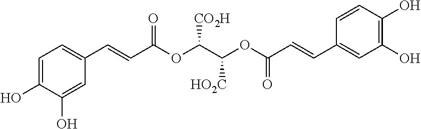 [SMILES: O=C(/C=C/c1ccc(O)c(O)c1)O[C@H](C(=O)O)[C@H](OC(=O)/C=C/c1ccc(O)c(O)c1)C(=O)O]

which has two caffeic acid molecules linked to a tartaric acid core; and caftaric acid having the structure:

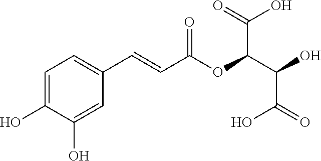 [SMILES: O=C(/C=C/c1ccc(O)c(O)c1)O[C@@H](C(=O)O)[C@@H](O)C(=O)O]

which has one caffeic acid molecule linked to a tartaric acid core.

Examples of the esters of the various acids contemplated herein include the ester of caffeic acid and 3-(3,4-dihydroxyphenyl)lactic acid including, for example, rosmarinic acid, which has the structure:

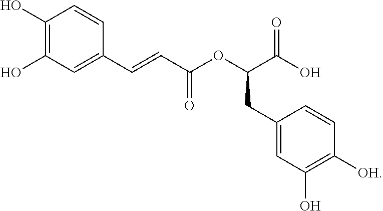 [SMILES: O=C(/C=C/c1ccc(O)c(O)c1)O[C@H](Cc1ccc(O)c(O)c1)C(=O)O]

Each of the caffeic acid, monocaffeoylquinic acids, dicaffeoylquinic acids and other surface tension reducing compounds can be considered weak acids and can each exist in at least one of their conjugate acid form, conjugate base form (e.g., in their salt form), and mixed conjugate acid-conjugate base form, wherein a fraction (e.g., mole fraction) of the compounds exist in the conjugate acid form and another fraction exist in the conjugate base form. The fraction of conjugate acid form to conjugate base form for the caffeic acid, monocaffeoylquinic acids, dicaffeoylquinic acids, and other surface tension reducing compounds will depend on various factors, including the pKa of each compound and the pH of the composition.

Examples of salts of caffeic acid, monocaffeoylquinic acids, dicaffeoylquinic acids, and other surface tension reducing compounds include, but are not limited to, quaternary ammonium, sodium, potassium, lithium, magnesium, and calcium salts of caffeic acid, monocaffeoylquinic acids, dicaffeoylquinic acids, monoferuloylquinic acids, and diferuloylquinic acids, and other surface tension reducing compounds and the like.

In some aspects, the surface tension reducing compound can be enriched for one or more of caffeic acid, monocaffeoylquinic acids, and dicaffeoylquinic acids. The term “enriched” refers to an increase in an amount of one of caffeic acid, monocaffeoylquinic acids, and dicaffeoylquinic acids relative to one or more other compounds that are present in the surface tension reducing compound. A surface tension reducing compound that is enriched for one or more of caffeic acid, monocaffeoylquinic acids, and dicaffeoylquinic acids can reduce surface tension of the steviol glycoside composition with reduced surface tension.

In some aspects, a surface tension reducing compound enriched for one or more dicaffeoylquinic acids can reduce surface tension of the steviol glycoside composition with reduced surface tension. A surface tension reducing compound that is enriched for dicaffeoylquinic acids can comprise 10% or more, 15% or more, 20% or more, 25% or more, 30% or more, 35% or more, 40% or more, 45% or more, or 50% or more, 60% or more, 70% or more, or 80% or more, or 90% or more dicaffeoylquinic acids. In other aspects, a surface tension reducing compound that is enriched for dicaffeoylquinic acids can comprise 10% or more, 15% or more, 20% or more, 25% or more, 30% or more, 35% or more, 40% or more, 45% or more, or 50% or more, 60% or more, 70% or more, or 80% or more, or 90% or more of a combination of one or more of 1,3-dicaffeoylquinic acid, 1,4-dicaffeoylquinic acid, 1,5-dicaffeoylquinic acid, 3,4-dicaffeoylquinic, 3,5-dicaffeoylquinic acid, and 4,5-dicaffeoylquinic acid, and salts thereof.

The amount of surface tension reducing compound in the steviol glycoside composition with reduced surface tension can vary. Surface tension reducing compound can be present in the steviol glycoside composition with reduced surface tension in any amount desired for the particular use. For example, surface tension reducing compound can be present in the steviol glycoside composition with reduced surface tension at from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, surface tension reducing compound can be present in the steviol glycoside composition with reduced surface tension at about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, surface tension reducing compound can be present in the steviol glycoside composition with reduced surface tension at from about 1000 ppm to about 5000 ppm, about 2000 ppm to about 5000 ppm, about 3000 ppm to about 5000 ppm, or about 4000 ppm to about 5000 ppm. In some aspects, surface tension reducing compound can be present in the steviol glycoside composition with reduced surface tension at or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, surface tension reducing compound can be present in the steviol glycoside composition with reduced surface tension at or greater than about 200000 ppm. In some aspects, surface tension reducing compound can be present in the steviol glycoside composition with reduced surface tension at or greater than about 300000 ppm. Unless otherwise expressly stated, ppm is on a by weight basis.

The amount of an individual surface tension reducing compound species in the steviol glycoside composition with reduced surface tension can vary. For example, an individual surface tension reducing compound species can be present in the steviol glycoside composition with reduced surface tension at a concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, an individual surface tension reducing compound species can be present in the steviol glycoside composition with reduced surface tension at a concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, an individual surface tension reducing compound species can be present in the steviol glycoside composition with reduced surface tension at a concentration from about 1000 ppm to about 5000 ppm, about 2000 ppm to about 5000 ppm, about 3000 ppm to about 5000 ppm, or about 4000 ppm to about 5000 ppm. In some aspects, an individual surface tension reducing compound species can be present in the steviol glycoside composition with reduced surface tension at a concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, an individual surface tension reducing compound species can be present in the steviol glycoside composition with reduced surface tension at a concentration of or greater than about 200000 ppm. In some aspects, an individual surface tension reducing compound species can be present in the steviol glycoside composition with reduced surface tension at a concentration of or greater than about 300000 ppm. Unless otherwise expressly stated, ppm is on a by weight basis

The amount of an individual surface tension reducing compound species in the steviol glycoside composition with reduced surface tension can vary. For example, dicaffeoylquinic acid can be present in the steviol glycoside composition with reduced surface tension at a concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, dicaffeoylquinic acid can be present in the steviol glycoside composition with reduced surface tension at a concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, dicaffeoylquinic acid can be present in the steviol glycoside composition with reduced surface tension at a concentration from about 1000 ppm to about 5000 ppm, about 2000 ppm to about 5000 ppm, about 3000 ppm to about 5000 ppm, or about 4000 ppm to about 5000 ppm. In some aspects, dicaffeoylquinic acid can be present in the steviol glycoside composition with reduced surface tension at a concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, dicaffeoylquinic acid can be present in the steviol glycoside composition with reduced surface tension at a concentration of or greater than about 200000 ppm. In some aspects, dicaffeoylquinic acid can be present in the steviol glycoside composition with reduced surface tension at a concentration of or greater than about 300000 ppm. Unless otherwise expressly stated, ppm is on a by weight basis.

In some aspects, the surface tension reducing compound may be isolated from botanical sources. Various botanical sources comprise surface tension reducing compounds and surface tension reducing compounds can be isolated from these plants. Some examples of botanical sources from which surface tension reducing compounds can be isolated include eucommoia ulmoides, honeysuckle, nicotiana benthamiana, globe artichoke, cardoon, Stevia, Stevia Rebaudiana, monkfruit, coffee, coffee beans, green coffee beans, tea, white tea, yellow tea, green tea, oolong tea, black tea, red tea, post-fermented tea, bamboo, heather, sunflower, blueberries, cranberries, bilberries, grouseberries, whortleberry, lingonberry, cowberry, huckleberry, grapes, chicory, eastern purple coneflower, echinacea, Eastern pellitory-of-the-wall, Upright pellitory, Lichwort, Greater celandine, Tetterwort, Nipplewort, Swallowwort, Bloodroot, Common nettle, Stinging nettle, Potato, Potato leaves, Eggplant, Aubergine, Tomato, Cherry tomato, Bitter apple, Thorn apple, Sweet potato, apple, Peach, Nectarine, Cherry, Sour cherry, Wild cherry, Apricot, Almond, Plum, Prune, Holly, Yerba mate, Mate, Ilex paraguariensis, Guayusa, Yaupon Holly, Kuding, Guarana, Cocoa, Cocoa bean, Cacao, Cacao bean, Kola nut, Kola tree, Cola nut, Cola tree, Hornwort, Ostrich fern, Oriental ostrich fern, Fiddlehead fern, Shuttlecock fern, Oriental ostrich fern, Asian royal fern, Royal fern, Bracken, Brake, Common bracken, Eagle fern, Eastern brakenfern, dandelion, algae, seagrasses, Clove, Cinnamon, Indian bay leaf, Nutmeg, Bay laurel, Bay leaf, Basil, Great basil, Saint-Joseph's-wort, Thyme, Sage, Garden sage, Common sage, Culinary sage, Rosemary, Oregano, Wild marjoram, Marjoram, Sweet marjoram, Knotted marjoram, Pot marjoram, Dill, Anise, Star anise, Fennel, Florence fennel, Tarragon, Estragon, Mugwort, Licorice, Liquorice, Soy, Soybean, Soyabean, Soya vean, Wheat, Common wheat, Rice, Canola, Broccoli, Cauliflower, Cabbage, Bok choy, Kale, Collard greens, Brussels sprouts, Kohlrabi, Winter's bark, Elderflower, Assa-Peixe, Greater burdock, Valerian, and Chamomile. In some aspects, the botanical source is yerba mate, chicory, rosemary, and/or Stevia.

Some plants may produce surface tension reducing compounds that are enriched for one or more of caffeic acid, monocaffeoylquinic acids, and dicaffeoylquinic acids and can reduce surface tension of the steviol glycoside composition with reduced surface tension. For example, surface tension reducing compounds isolated from yerba mate plant (Ilex paraguariensis) are enriched for dicaffeoylquinic acids and can reduce surface tension of the steviol glycoside composition with reduced surface tension. In other aspects, surface tension reducing compounds isolated from yerba mate plant that are enriched for dicaffeoylquinic acids can comprise 10% or more, 15% or more, 20% or more, 25% or more, 30% or more, 35% or more, 40% or more, 45% or more, or 50% or more, 60% or more, 70% or more, or 80% or more, or 90% or more of a combination of one or more of 1,3-dicaffeoylquinic acid, 1,4-dicaffeoylquinic acid, 1,5-dicaffeoylquinic acid, 3,4-dicaffeoylquinic, 3,5-dicaffeoylquinic acid, and 4,5-dicaffeoylquinic acid, and salts thereof. For example, surface tension reducing compounds isolated from other plants can be enriched for dicaffeoylquinic acids and can reduce surface tension of the steviol glycoside composition with reduced surface tension. In other aspects, surface tension reducing compounds isolated from other plants that are enriched for dicaffeoylquinic acids can comprise 10% or more, 15% or more, 20% or more, 25% or more, 30% or more, 35% or more, 40% or more, 45% or more, or 50% or more, 60% or more, 70% or more, or 80% or more, or 90% or more of a combination of one or more of 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid, and salts thereof.

In some aspects, the surface tension reducing compound can be a blend of surface tension reducing compound isolated from more than one botanical source. The surface tension reducing compound can be a blend of surface tension reducing compound isolated from more than one botanical source.

In some aspects, the ratio of surface tension reducing compound to steviol glycoside in the steviol glycoside composition with reduced surface tension can vary. The ratio of surface tension reducing compound to steviol glycoside in the steviol glycoside composition with reduced surface tension can be in any amount effective to reduce surface tension. For example, the ratio of surface tension reducing compound to steviol glycoside can be from about 0.1:1 to 10:1. In some aspects, the ratio of surface tension reducing compound to steviol glycoside can be in the range of about 0.1:1 to 5:1, about 0.5:1 to 4:1, or about 1:1 to 3:1. In other aspects, the ratio of surface tension reducing compound to steviol glycoside is about 1:1, 2:1, 3:1, 4:1, 5:1, 6:1, 7:1, 8:1, 9:1, or 10:1. In other aspects, the ratio of surface tension reducing compound to steviol glycoside is about 0.1:1, 0.2:1, 0.3:1, 0.4:1, 0.5:1, 0.6:1, 0.7:1, 0.8:1, or 0.9:1. In other aspects, the ratio of surface tension reducing compound to steviol glycoside is about 1.1:1, 1.2:1, 1.3:1, 1.4:1, 1.5:1, 1.6:1, 1.7:1, 1.8:1, or 1.9:1. In other aspects, the ratio of surface tension reducing compound to steviol glycoside is about 2.1:1, 2.2:1, 2.3:1, 2.4:1, 2.5:1, 2.6:1, 2.7:1, 2.8:1, or 2.9:1. In other aspects, the ratio of surface tension reducing compound to steviol glycoside is about 3.1:1, 3.2:1, 3.3:1, 3.4:1, 3.5:1, 3.6:1, 3.7:1, 3.8:1, or 3.9:1. In other aspects, the ratio of surface tension reducing compound to steviol glycoside is about 4.1:1, 4.2:1, 4.3:1, 4.4:1, 4.5:1, 4.6:1, 4.7:1, 4.8:1, or 4.9:1.

In some aspects, the steviol glycoside composition with reduced surface tension can comprise an surface tension reducing compound in an amount effective to reduce the surface tension compared to a steviol glycoside solution without an surface tension reducing compound. This reduction in surface tension can be at least 1%, at least 2%, at least 3%, at least 4%, at least 5%, at least 6%, at least 7%, at least 8%, at least 9%, at least 10%, at least 11%, at least 12%, at least 13%, at least 14%, or at least 15%. The reduction in surface tension can be at least 20%, 30%, 40%, 50%, 60%, 70%, 80%, or 90%. The reduction in surface tension can refer to a reduction in initial surface tension of the steviol glycoside composition with reduced surface tension comprising an surface tension reducing compound compared to similar a similar steviol glycoside composition without the surface tension reducing compound. The reduction in surface tension can also refer to a reduction in surface tension of the steviol glycoside composition with reduced surface tension comprising an surface tension reducing compound compared to similar a similar steviol glycoside composition without the surface tension reducing compound over time. The reduction in surface tension can also refer to a reduction in surface tension of the steviol glycoside composition with reduced surface tension comprising an surface tension reducing compound compared to similar a similar steviol glycoside composition without the surface tension reducing compound at equilibrium.

In some aspects, surface tension can be measured by various methods known to the skilled person. A tensiometer can be used to measure surface tension by various methods, including, but not limited to Du Noüy ring method, Du Noüy-Padday method, Wilhelmy plate method, spinning drop method, pendant drop method, bubble pressure method, drop volume method, capillary rise method, stalagmometric method, sessile drop method, vibrational frequency of levitated drops, resonant oscillations of spherical and hemispherical liquid drop.

In some aspects, the steviol glycoside composition with reduced surface tension can comprise additives, flavors, colors, fillers, bulking agents, and other additives including, but not limited to, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, weighing agents, gums, antioxidants, colorants, flavonoids, alcohols, polymers and combinations thereof, caffeine, quinine, urea, bitter orange oil, naringin, quassia, and salts thereof, vanillin, vanilla extract, mango extract, cinnamon, citrus, coconut, ginger, viridiflorol, almond, menthol (including menthol without mint), cola, lemon, lime, root beer, sarsaparilla, orange, grape skin extract, grape seed extract, chitosan, pectin, pectic, pectinic, polyuronic, polygalacturonic acid, starch, food hydrocolloid or crude extracts thereof (e.g., gum acacia Senegal (Fibergum™), gum acacia seyal, carageenan), poly-L-lysine (e.g., poly-L-a-lysine or poly-L-e-lysine), poly-L-ornithine (e.g., poly-L-a-ornithine or poly-L-e-ornithine), polypropylene glycol, polyethylene glycol, poly(ethylene glycol methyl ether), polyarginine, polyaspartic acid, polyglutamic acid, polyethylene imine, alginic acid, sodium alginate, propylene glycol alginate, and sodium polyethyleneglycolalginate, sodium hexametaphosphate and its salts, and other cationic polymers and anionic polymers. In some aspects, one or more additives is present in the steviol glycoside composition with reduced surface tension at about 0.1 ppm to about 4,000 ppm.

An example of a steviol glycoside composition with reduced surface tension includes a beverage concentrate solution with reduced surface tension comprising a steviol glycoside and an surface tension reducing compound in an amount effective to reduce surface tension by at least 10% during at least six-fold dilution of the concentrate solution. Another example of a beverage concentrate solution with reduced surface tension comprises a steviol glycoside and a surface tension reducing compound in an amount effective to reduce surface tension by at least 1%, at least 2%, at least 3%, at least 4%, at least 5%, at least 6%, at least 7%, at least 8%, at least 9%, at least 10%, at least 11%, at least 12%, at least 13%, at least 14%, or at least 15% during at least six-fold dilution of the concentrate solution. Another example of a beverage concentrate solution with reduced surface tension comprises a steviol glycoside and an surface tension reducing compound in an amount effective to reduce surface tension by at least 20%, 30%, 40%, 50%, 60%, 70%, 80%, or 90% during at least six-fold dilution of the concentrate solution.

In some aspects, the beverage concentrate solution with reduced surface tension comprises a throw syrup for preparing a carbonated soda drink. The beverage concentrate solution with reduced surface tension can also comprise other drink concentrates used to prepare beverages. In other aspects, the beverage concentrate solution with reduced surface tension comprises a throw syrup for preparing a carbonated soda drink in a soda fountain. In some aspects, the beverage concentrate solution with reduced surface tension comprises an aqueous solution. The aqueous solution can comprise primarily water. The aqueous solution can also comprise a buffer such as a citrate/phosphate buffer. The citrate/phosphate buffer can have a pH of 1.5 to 4.

In some aspects, the beverage concentrate solution with reduced surface tension is diluted before use as a beverage. For example, the beverage concentrate solution with reduced surface tension can be diluted in a soda fountain by diluting with a stream of carbonated water as the beverage is dispensed. This diluting of the beverage concentrate solution with reduced surface tension with the stream of carbonated water can dilute the beverage concentrate solution with reduced surface tension by about 6 to 7 times. The surface tension reducing compound can be present in the beverage concentrate solution with reduced surface tension in an effective amount to reduce surface tension during this dilution as the beverage is dispensed. In some aspects, the beverage concentrate solution with reduced surface tension comprises a total steviol glycoside concentration of about 2400 to 4200 ppm. In other aspects, the beverage concentrate solution with reduced surface tension comprises an surface tension reducing compound concentration of 1800 to 5400 ppm. In some aspects, the beverage concentrate solution with reduced surface tension comprises an surface tension reducing compound concentration of 1800 to 1000 ppm.

The beverage concentrate solution with reduced surface tension can also be diluted by mixing with water (or carbonated water) before use as a beverage by a consumer. For example, the beverage concentrate solution with reduced surface tension can be diluted with water or other similar solution such as carbonated water and then mixed. The surface tension reducing compound can be present in the beverage concentrate solution with reduced surface tension in an effective amount to reduce surface tension during the dilution and mixing of the beverage. In some aspects, the beverage concentrate solution with reduced surface tension comprises a total steviol glycoside concentration of about 2400 to 4200 ppm. In other aspects, the beverage concentrate solution with reduced surface tension comprises an surface tension reducing compound concentration of about 1800 to 5400 ppm. In other aspects, the beverage concentrate solution with reduced surface tension comprises an surface tension reducing compound concentration of about 1800 to 1000 ppm.

An example of a steviol glycoside composition with reduced surface tension comprises a dry admixture of steviol glycoside and surface tension reducing compound, wherein an aqueous solution prepared from the dry admixture comprises reduced surface tension compared to an aqueous solution prepared from a dry admixture of steviol glycoside without surface tension reducing compound. In some aspects, surface tension is reduced by at least 10%. In other aspects, surface tension is reduced by at least 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%, 75%, 80%, 85%, 90%, 95% or more. In some aspects, initial surface tension is reduced by at least 10%. In other aspects, initial surface tension is reduced by at least 10%, 15%, 20%, 25%, 30%, 35%, 40%, 45%, 50%, 55%, 60%, 65%, 70%, 75%, 80%, 85%, 90%, 95% or more. In some aspects, surface tension decreases more quickly over time by at least 10%. In other aspects, surface tension decreases more quickly over time by at least 10%, 15%, 20%, 25%, 30%, 35%, 40%, 45%, 50%, 55%, 60%, 65%, 70%, 75%, 80%, 85%, 90%, 95% or more.

In some aspects, an example of a steviol glycoside composition with reduced surface tension comprises a dry admixture of steviol glycoside and surface tension reducing compound, wherein an aqueous solution prepared from the dry admixture has a reduced surface tension compared to a steviol glycoside solution without surface tension reducing compound. This reduction in surface tension can be at least 1%, at least 2%, at least 3%, at least 4%, at least 5%, at least 6%, at least 7%, at least 8%, at least 9%, at least 10%, at least 11%, at least 12%, at least 13%, at least 14%, or at least 15%. The reduction in surface tension can be at least 20%, 30%, 40%, 50%, 60%, 70%, 80%, or 90%.

In some aspects, the dry admixture is prepared with an aqueous solution comprising primarily water. The aqueous solution can also be buffered with any suitable buffering system, including, but not limited to, one or more buffers such as a phosphate, a citrate, ascorbate, lactate, acetate, and the like. The buffer can comprise 1-1000 mM of the anion component. In other aspects, the aqueous solution comprises a citrate/phosphate buffer. In some aspects, citrate/phosphate buffer can have a pH of 2 to 4.

The amounts of steviol glycoside in the dry admixture and the surface tension reducing compound can be expressed in relation to one another, or to the total amount of steviol glycosides (TSG), such as a weight percentage of the total amount of steviol glycosides (TSG), or to the total amount of surface tension reducing compound, or a ratio, or range of ratios, expressed as a weight percent, or molar percent.

The dry admixture can include one or more steviol glycosides. In some aspects, the term steviol glycoside refers to Rebaudioside A (Reb A) (CAS #58543-16-1), Rebaudioside B (Reb B) (CAS #58543-17-2), Rebaudioside C (Reb C) (CAS #63550-99-2), Rebaudioside D (Reb D) (CAS #63279-13-0), Rebaudioside E (Reb E) (CAS #63279-14-1), Rebaudioside F (Reb F) (CAS #438045-89-7), Rebaudioside M (Reb M) (CAS #1220616-44-3), Rubusoside (CAS #63849-39-4), Dulcoside A (CAS #64432-06-0), Rebaudioside I (Reb I) (MassBank Record: FU000332), Rebaudioside Q (Reb Q), Rebaudioside O (Reb O), Rebaudioside N (Reb N) (CAS #1220616-46-5), 1,2-Stevioside (CAS #57817-89-7), 1,3-Stevioside (Reb G), Steviol-1,2-Bioside (MassBank Record: FU000299), Steviol-1,3-Bioside, Steviol-13-O-glucoside (13-SMG), Steviol-19-O-glucoside (19-SMG), and steviol glycoside having 1, 2, 3, 4, 5, 6, 7, 8, 9, 10 or sugar additions (e.g., glucose, rhamnose, and/or xylose), and isomers thereof. See FIG. 1; see also, Steviol Glycosides Chemical and Technical Assessment 69th JECFA, 2007, prepared by Harriet Wallin, Food Agric. Org.

In some aspects, the dry admixture can include rebaudioside A. For example, the dry admixture can include blends of rebaudioside A and one or more other steviol glycosides. An example of a blend of rebaudioside A with one or more other steviol glycosides comprises 60% or more rebaudioside A (RA60). Another example of a blend of rebaudioside A with one or more other steviol glycosides comprises 80% or more rebaudioside A (RA80). Another example of a blend of rebaudioside A with one or more other steviol glycosides comprises 95% or more rebaudioside A (RA95). In other aspects, steviol glycosides can include blends of one or more of RebA, RebB, RebC, RebD, RebE, RebF, RebM, rubusoside, dulcoside A, RebI, RebQ, 1,2 stevioside, 1,3 stevioside, steviol-1,2-bioside, steviol-1,3-bioside, 13-SMG, 19-SMG, a tri-glycosylated steviol glycoside, a tetra-glycosylated steviol glycoside, a penta-glycosylated steviol glycoside, a hexa-glycosylated steviol glycoside, a hepta-glycosylated steviol glycoside, and isomers thereof.

In some aspects, the dry admixture can comprise blends of rebaudioside M and rebaudioside D. For example, rebaudioside M and rebaudioside D can be present in the blend in a total amount of about 80% (wt) or greater, 90% (wt) or greater, or 95% (wt) or greater, of a total amount steviol glycosides (TSG) in the composition. Rebaudioside M can be the predominant steviol glycoside in the blend, and can be present, for example, in an amount in the range of about 50% to about 95%, about 70% to about 90%, or about 75% to about 85% of the total amount steviol glycosides (TSG) in the composition. Rebaudioside D can be in an amount less than Rebaudioside M, such as in an amount in the range of about 5% to about 25%, about 10% to about 20%, or about 10% to about 15% of the total amount of steviol glycosides (TSG) in the composition.

The amount of steviol glycosides in the dry admixture can vary. Steviol glycosides can be present in the dry admixture in any amount desired for the particular use. For example, steviol glycosides can be present in the dry admixture at a total concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, steviol glycosides can be present in the dry admixture at a total concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, steviol glycosides can be present in the dry admixture at a total concentration from about 1000 ppm to about 5000 ppm, about 2000 ppm to about 5000 ppm, about 3000 ppm to about 5000 ppm, or about 4000 ppm to about 5000 ppm. In some aspects, steviol glycosides can be present in the dry admixture at a total concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, steviol glycosides can be present in the dry admixture at a total concentration of or greater than about 200000 ppm. In some aspects, steviol glycosides can be present in the dry admixture at a total concentration of or greater than about 300000 ppm. Unless otherwise expressly stated, ppm is on a by weight basis.

The amount of steviol glycoside in the dry admixture can vary. For example, an individual steviol glycoside can be present in the dry admixture at a concentration of about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more.

The amount of an individual steviol glycoside species in the dry admixture can vary. For example, an individual steviol glycoside species can be present in the dry admixture at a concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, an individual steviol glycoside species can be present in the dry admixture at a concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, an individual steviol glycoside species can be present in the dry admixture at a concentration from about 1000 ppm to about 5000 ppm, about 2000 ppm to about 5000 ppm, about 3000 ppm to about 5000 ppm, or about 4000 ppm to about 5000 ppm. In some aspects, an individual steviol glycoside species can be present in the dry admixture at a concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, an individual steviol glycoside species can be present in the dry admixture at a concentration of or greater than about 200000 ppm. In some aspects, an individual steviol glycoside species can be present in the dry admixture at a concentration of or greater than about 300000 ppm. Unless otherwise expressly stated, ppm is on a by weight basis

The amount of an individual steviol glycoside in the dry admixture can vary. For example, an individual steviol glycoside can be present in the dry admixture at a concentration of about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more.

The amount of an individual steviol glycoside species in the dry admixture composition can vary. For example, RebA can be present in the dry admixture at a concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, RebA can be present in the dry admixture at a concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, RebA can be present in the dry admixture at a concentration from about 1000 ppm to about 5000 ppm, about 2000 ppm to about 5000 ppm, about 3000 ppm to about 5000 ppm, or about 4000 ppm to about 5000 ppm. In some aspects, RebA can be present in the dry admixture at a concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, RebA can be present in the dry admixture at a concentration of or greater than about 200000 ppm. In some aspects, RebA can be present in the dry admixture at a concentration of or greater than about 300000 ppm. Unless otherwise expressly stated, ppm is on a by weight basis.

The amount of an individual steviol glycoside species in the dry admixture can vary. For example, RebM can be present in the dry admixture at a concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, RebM can be present in the dry admixture at a concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, RebM can be present in the dry admixture at a concentration from about 1000 ppm to about 5000 ppm, about 2000 ppm to about 5000 ppm, about 3000 ppm to about 5000 ppm, or about 4000 ppm to about 5000 ppm. In some aspects, RebM can be present in the dry admixture at a concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, RebM can be present in the dry admixture at a concentration of or greater than about 200000 ppm. In some aspects, RebM can be present in the dry admixture at a concentration of or greater than about 300000 ppm. Unless otherwise expressly stated, ppm is on a by weight basis.

In some aspects, the dry admixture comprises steviol glycoside at 1%, 2%, 3%, 4%, 5%, 6%, 7%, 8%, 9%, 10% or more by weight. The dry admixture can comprise steviol glycoside at 11%, 12%, 13%, 14%, 15%, 16%, 17%, 18%, 19%, 20% or more by weight. The dry mixture can comprise steviol glycoside at 21%, 22%, 23%, 24%, 25%, 26%, 27%, 28%, 29%, 30% or more by weight. The dry mixture can comprise steviol glycoside at 35%, 40%, 45%, 50%, 55%, 60%, 65%, 70%, 75%, 80%, 85%, 90% or more by weight.

The amount of surface tension reducing compound in the dry admixture can vary. Surface tension reducing compound can be present in the dry admixture in any amount desired for the particular use. For example, surface tension reducing compound can be present in the dry admixture at about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, surface tension reducing compound can be present in the dry admixture at about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, surface tension reducing compound can be present in the dry admixture at about 1000 ppm to about 5000 ppm, about 2000 ppm to about 5000 ppm, about 3000 ppm to about 5000 ppm, or about 4000 ppm to about 5000 ppm. In some aspects, surface tension reducing compound can be present in the dry admixture at or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, surface tension reducing compound can be present in the dry admixture at or greater than about 200000 ppm. In some aspects, surface tension reducing compound can be present in the dry admixture at or greater than about 300000 ppm. Unless otherwise expressly stated, ppm is on a by weight basis.

The amount of surface reducing compound in the dry admixture can vary. For example, an individual steviol glycoside can be present in the dry admixture at a concentration of about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more.

The amount of an individual surface tension reducing compound species in the dry admixture composition can vary. For example, an individual surface tension reducing compound can be present in the dry admixture at a concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, an individual surface tension reducing compound species can be present in the dry admixture at a concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, an individual surface tension reducing compound species can be present in the dry admixture at a concentration from about 1000 ppm to about 5000 ppm, about 2000 ppm to about 5000 ppm, about 3000 ppm to about 5000 ppm, or about 4000 ppm to about 5000 ppm. In some aspects, an individual surface tension reducing compound species can be present in the dry admixture at a concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, an individual surface tension reducing compound species can be present in the dry admixture at a concentration of or greater than about 200000 ppm. In some aspects, an individual surface tension reducing compound species can be present in the dry admixture at a concentration of or greater than about 300000 ppm. Unless otherwise expressly stated, ppm is on a by weight basis

In some aspects, the surface tension reducing compound in the dry admixture can be enriched for one or more of caffeic acid, monocaffeoylquinic acids, and dicaffeoylquinic acids. The term “enriched” refers to an increase in an amount of one of caffeic acid, monocaffeoylquinic acids, and dicaffeoylquinic acids relative to one or more other compounds that are present in the surface tension reducing compound. An surface tension reducing compound that is enriched for one or more of caffeic acid, monocaffeoylquinic acids, and dicaffeoylquinic acids can reduce surface tension of the steviol glycoside composition with reduced surface tension. A surface tension reducing compound that is enriched for dicaffeoylquinic acids can comprise 10% or more, 15% or more, 20% or more, 25% or more, 30% or more, 35% or more, 40% or more, 45% or more, or 50% or more, 60% or more, 70% or more, or 80% or more, or 90% or more dicaffeoylquinic acids. In other aspects, a surface tension reducing compound that is enriched for dicaffeoylquinic acids can comprise 10% or more, 15% or more, 20% or more, 25% or more, 30% or more, 35% or more, 40% or more, 45% or more, or 50% or more, 60% or more, 70% or more, or 80% or more, or 90% or more of a combination of one or more of 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid, and salts thereof.

The amount of an individual surface tension reducing compound species in the admixture can vary. For example, dicaffeoylquinic acid can be present in the admixture at a concentration from about 1 ppm to about 1000 ppm, from about 1 ppm to about 10000 ppm, from about 1 ppm to about 100000 ppm, from about 1 ppm to about 200000 ppm, or from about 1 ppm to about 300000 ppm. In some aspects, dicaffeoylquinic acid can be present in the admixture at a concentration from about 100 ppm to about 5000 ppm, about 200 ppm to about 5000 ppm, 300 ppm to about 5000 ppm, 400 ppm to about 5000 ppm, 500 ppm to about 5000 ppm, 600 ppm to about 5000 ppm, 700 ppm to about 5000 ppm, 800 ppm to about 5000 ppm, 900 ppm to about 5000 ppm, or 1000 ppm to about 5000 ppm. In some aspects, dicaffeoylquinic acid can be present in the admixture at a concentration from about 1000 ppm to about 5000 ppm, about 2000 ppm to about 5000 ppm, about 3000 ppm to about 5000 ppm, or about 4000 ppm to about 5000 ppm. In some aspects, dicaffeoylquinic acid can be present in the admixture at a concentration of or greater than about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 ppm. In some aspects, dicaffeoylquinic acid can be present in the admixture at a concentration of or greater than about 200000 ppm. In some aspects, dicaffeoylquinic acid can be present in the admixture at a concentration of or greater than about 300000 ppm. Unless otherwise expressly stated, ppm is on a by weight basis.

The amount of an individual surface tension reducing compound species in the dry admixture can vary. For example, an individual steviol glycoside species can be present in the dry admixture at a concentration of about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more.

In some aspects, the dry admixture comprises surface tension reducing compound at 1%, 2%, 3%, 4%, 5%, 6%, 7%, 8%, 9%, 10% or more by weight. The dry admixture can comprise surface tension reducing compound at 11%, 12%, 13%, 14%, 15%, 16%, 17%, 18%, 19%, 20% or more by weight. The dry mixture can comprise surface tension reducing compound at 21%, 22%, 23%, 24%, 25%, 26%, 27%, 28%, 29%, 30% or more by weight. The dry mixture can comprise surface tension reducing compound at 35%, 40%, 45%, 50%, 55%, 60%, 65%, 70%, 75%, 80%, 85%, 90% or more by weight.

In some aspects, the ratio of surface tension reducing compound to steviol glycoside in the dry admixture can vary. The ratio of surface tension reducing compound to steviol glycoside in the dry admixture can be in any amount effective to reduce surface tension. For example, the ratio of surface tension reducing compound to steviol glycoside can be from about 0.1:1 to 10:1. In some aspects, the ratio of surface tension reducing compound to steviol glycoside can be in the range of about 0.1:1 to 5:1, about 0.5:1 to 4:1, or about 1:1 to 3:1. In other aspects, the ratio of surface tension reducing compound to steviol glycoside is about 1:1, 2:1, 3:1, 4:1, 5:1, 6:1, 7:1, 8:1, 9:1, or 10:1. In other aspects, the ratio of surface tension reducing compound to steviol glycoside is about 0.1:1, 0.2:1, 0.3:1, 0.4:1, 0.5:1, 0.6:1, 0.7:1, 0.8:1 or 0.9:1. In other aspects, the ratio of surface tension reducing compound to steviol glycoside is about 1.1:1, 1.2:1, 1.3:1, 1.4:1, 1.5:1, 1.6:1, 1.7:1, 1.8:1, or 1.9:1. In other aspects, the ratio of surface tension reducing compound to steviol glycoside is about 2.1:1, 2.2:1, 2.3:1, 2.4:1, 2.5:1, 2.6:1, 2.7:1, 2.8:1, or 2.9:1. In other aspects, the ratio of surface tension reducing compound to steviol glycoside is about 3.1:1, 3.2:1, 3.3:1, 3.4:1, 3.5:1, 3.6:1, 3.7:1, 3.8:1, or 3.9:1. In other aspects, the ratio of surface tension reducing compound to steviol glycoside is about 4.1:1, 4.2:1, 4.3:1, 4.4:1, 4.5:1, 4.6:1, 4.7:1, 4.8:1, or 4.9:1.

In some aspects, the dry admixture can comprise additives, flavors, colors, fillers, bulking agents, and other additives including, but not limited to those described above. In other aspects, the one or more additives is present in the dry admixture at about 0.1 ppm to about 4,000 ppm. In some aspects, the one or more additives is present in the dry admixture at about 0.1% to about 99% by weight.

In some aspects, the present disclosure is drawn to methods for reducing surface tension in a steviol glycoside solution. For example, a method for reducing surface tension in a steviol glycoside solution can comprise preparing any of the steviol glycoside composition with reduced surface tensions described above. A method for reducing surface tension in a steviol glycoside solution can comprise contacting a steviol glycoside composition and an surface tension reducing compound with an aqueous solution to prepare any of the steviol glycoside composition with reduced surface tensions described above, In other aspects, a method for reducing surface tension in a steviol glycoside solution can comprise contacting a steviol glycoside solution and surface tension reducing solution to prepare one of the steviol glycoside composition with reduced surface tensions described above. In some aspects, a method for reducing surface tension in a steviol glycoside solution can comprise contacting a steviol glycoside solution with dry surface tension reducing compound to prepare one of the steviol glycoside composition with reduced surface tensions described above. Additionally, a method for reducing surface tension in a steviol glycoside solution can comprise contacting a dry steviol glycoside with an surface tension reducing solution to prepare one of the steviol glycoside composition with reduced surface tensions described above. Also, a method for reducing surface tension in a steviol glycoside solution can comprise preparing any of the admixtures of steviol glycoside and surface tension reducing compound as described above and then preparing an aqueous solution using the admixture as described above.

In some aspects, a method for reducing surface tension in a steviol glycoside solution can reduce surface tension by at least 10%, at least 20%, at least 30%, at least 40%, at least 50%, at least 60%, at least 70%, at least 80% or at least 90% compared to a steviol glycoside solution without an surface tension reducing compound. In some aspects, a method for reducing surface tension in a steviol glycoside solution can reduce surface tension by at least 1%, 2%, 3%, 4%, 5%, 6%, 7%, 8%, 9%, 10%, 11%, 12%, 13%, 14%, 15%, 16%, 17%, 18%, 19% or 20% compared to a steviol glycoside solution without an surface tension reducing compound. In other aspects, a method for reducing surface tension in a steviol glycoside solution can reduce initial surface tension by at least 10%, at least 20%, at least 30%, at least 40%, at least 50%, at least 60%, at least 70%, at least 80% or at least 90% compared to a steviol glycoside solution without an surface tension reducing compound. In some aspects, a method for reducing surface tension in a steviol glycoside solution can reduce surface tension over time by at least 10%, at least 20%, at least 30%, at least 40%, at least 50%, at least 60%, at least 70%, at least 80% or at least 90% compared to a steviol glycoside solution without an surface tension reducing compound. In some aspects, a method for reducing surface tension in a steviol glycoside solution can reduce surface tension at equilibrium by at least 10%, at least 20%, at least 30%, at least 40%, at least 50%, at least 60%, at least 70%, at least 80% or at least 90% compared to a steviol glycoside solution without an surface tension reducing compound.

In some aspects, the present disclosure is drawn to methods for reducing surface tension in a steviol glycoside solution. For example, a method for reducing surface tension in a steviol glycoside solution can comprise preparing any of the steviol glycoside composition with reduced surface tensions described above. A method for reducing surface tension in a steviol glycoside solution can comprise contacting a steviol glycoside composition and an surface tension reducing compound with an aqueous solution to prepare any of the steviol glycoside composition with reduced surface tensions described above, In other aspects, a method for reducing surface tension in a steviol glycoside solution can comprise contacting a steviol glycoside solution and a surface tension reducing solution to prepare one of the steviol glycoside composition with reduced surface tensions described above. In some aspects, a method for reducing surface tension in a steviol glycoside solution can comprise contacting a steviol glycoside solution with dry surface tension reducing compound to prepare one of the steviol glycoside composition with reduced surface tensions described above. Additionally, a method for reducing surface tension in a steviol glycoside solution can comprise contacting a dry steviol glycoside with a surface tension reducing solution to prepare one of the steviol glycoside composition with reduced surface tensions described above. Also, a method for reducing surface tension in a steviol glycoside solution can comprise preparing any of the admixtures of steviol glycoside and surface tension reducing compound as described above and then preparing an aqueous solution using the admixture as described above.

In some aspects, a method for reducing surface tension in a steviol glycoside solution can reduce surface tension by at least 10%, at least 20%, at least 30%, at least 40%, at least 50%, at least 60%, at least 70%, at least 80% or at least 90% compared to a steviol glycoside solution without an surface tension reducing compound.

As used herein, the singular forms “a,” “an,” and “the” include plural referents unless the context clearly dictates otherwise. For example, reference to “a steviol glycoside” means one or more steviol glycosides.

### EXAMPLES

The following examples are provided to illustrate the disclosure, but are not intended to limit the scope thereof. All parts and percentages are by weight unless otherwise indicated.

### Example 1

A series of surface tension assays were carried out to characterize steviol glycoside compositions with and without surface tension reducing compound. A steviol glycoside blend comprising primarily rebaudioside M (RM80) was used. A surface tension reducing compound was prepared from yerba mate. The yerba mate surface tension reducing compound comprised about 50% dicaffeoylquinic acids. Chlorogenic acid was obtained commercially and comprised primarily monocaffeoylquinic acids.

A control solution of 1000 ppm of the RM80 was prepared and labelled as “A”. A control solution of 1000 ppm of the yerba mate surface tension reducing compound was prepared and labelled as “B”. A test solution of 1000 ppm of RM80 blend and 1000 ppm of yerba mate surface tension reducing compound was prepared and labelled as “C”. A test solution of 1000 ppm of RM80 and 3000 ppm of chlorogenic acid was prepared and labelled as “D”. Each solution was also observed visually for foaming. The control solution of 1000 ppm of the yerba mate surface tension reducing compound produced no foam. The test solution of 1000 ppm of RM80 and 1000 ppm of yerba mate surface tension reducing compound produced foam with smaller bubbles than the test solution of 1000 ppm of RM80 and 3000 ppm of chlorogenic acid.

Surface tension measurements of each of the control solutions and test solutions were determined by a Kruss K100MK2 Tensiometer with a roughened platinum and iridium Wilhelmy plate in a 45 mm diameter glass vessel at 20° C. The surface tension of the control solution of 1000 ppm of the RM80 (“A”) was 63.51 mN/m (n=2). The surface tension of the control solution of 1000 ppm of the yerba mate surface tension reducing compound (“B”) was 50.45 mN/m (n=3). The surface tension of the test solution of 1000 ppm of RM80 and 1000 ppm of yerba mate surface tension reducing compound (“C”) was 55.66 mN/m (n=2). The surface tension of the test solution of 1000 ppm of RM80 and 3000 ppm of chlorogenic acid (“D”) was 63.68 mN/m (n=2). The surface tension measurements are shown below in Table 1 and in FIG. 1.

TABLE 1

| | | | Surface tension |
|---|---|---|---|
| | RebD/ | Surface tension reducing | (mN/m at 20° at |
| Sample | RebM | compound | equilibrium) |
| A | 1000 ppm | — | 63.51 |
| B | — | 1000 ppm of yerba mate | 50.45 |
| | | surface tension | |
| | | reducing compound | |
| C | 1000 ppm | 1000 ppm of yerba mate | 55.66 |
| | | surface tension | |
| | | reducing compound | |
| D | 1000 ppm | 3000 ppm of commercially | 63.68 |
| | | prepared chlorogenic acid | |

The surface tension of the control solution of yerba mate surface tension reducing compound was lower than the surface tension of the control solution of RM80. The surface tension of the test solution of RM80 and yerba mate surface tension reducing compound was lower than the surface tension of the control solution of RM80. The surface tension of the test solution of RM80 and chlorogenic acid was similar to the control solution of RM80. The surface tension assays showed that the yerba mate surface tension reducing compound lowered the surface tension of the RM80 solution. The surface tension assays also showed that the chlorogenic acid likely did not affect the surface tension of the RM80 solution. The surface tension assays showed that the yerba mate surface tension reducing compound, which contained the higher ratio of dicaffeoylquinic acids (˜50%), was more effective in reducing the surface tension of the steviol glycoside solution than the chlorogenic acid which comprised primarily monocaffeoylquinic acids.

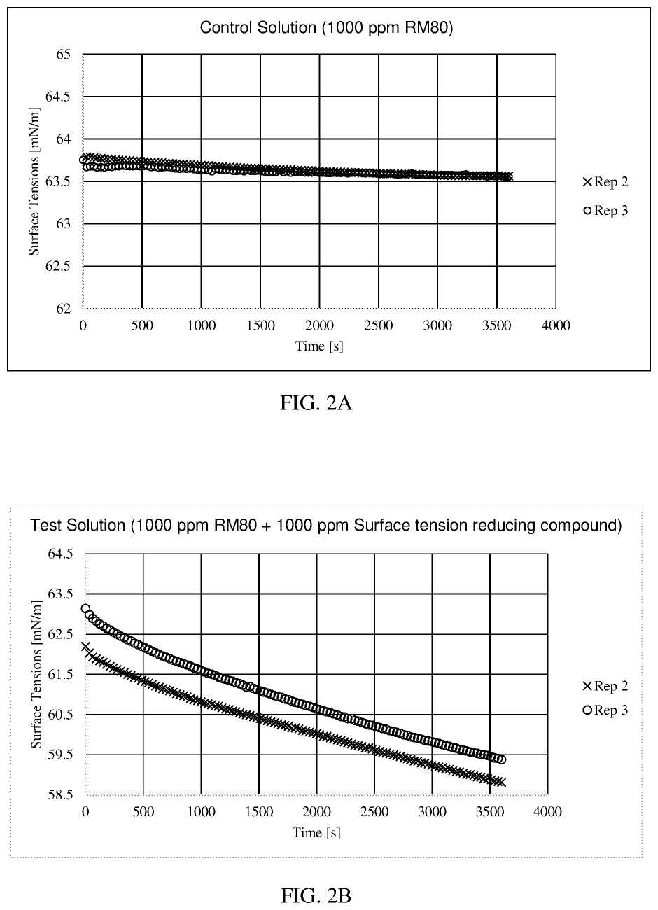

Kinetic surface plots of surface tension over time were also recorded for the control solution of 1000 ppm of the RM80 and the test solution of 1000 ppm of RM80 and 1000 ppm of yerba mate surface tension reducing compound. FIG. 2A shows the kinetic surface plot of surface tension over time for the control solution of 1000 ppm of the RM80 (“A”). FIG. 2B shows the kinetic surface plot of surface tension over time for the test solution of 1000 ppm of RM80 and 1000 ppm of yerba mate surface tension reducing compound (“C”). The test solution of 1000 ppm of RM80 and 1000 ppm of yerba mate surface tension reducing compound showed significant reduction of surface tension over time indicating active diffusion from the bulk solution to the interface layer. The control solution of 1000 ppm of the RM80 exhibited small dynamic ranges (e.g. <1 mN/m).

### Example 2

A series of surface tension assays were carried out to characterize steviol glycoside composition with and without surface tension reducing compounds. Highly purified rebaudioside A (˜95%), rebaudioside D (>99%), rebaudioside M (>99%) were assayed. Rebaudioside A blend derived from Stevia leaf at ≥80% rebaudioside A (RA80) was also used. A surface tension reducing compound was prepared from yerba mate. The yerba mate surface tension reducing compound comprised about 50% dicaffeoylquinic acids.

Control and test solutions were prepared by adding 200 mg of the respective steviol glycoside and/or 200 mg of the surface tension reducing compound to each individual container and then adding 400 ml of ultrapure (18.2 MSΩ) water. The individual containers were sealed and shaken to mix. The solutions containing only Reb D or only Reb M were heated to 40-50° C. to fully solubilize the steviol glycoside. The individual containers were allowed to equilibrate at room temperature and then stored at room temperature until assayed. The respective makeup of each solution is shown in Table 2.

TABLE 2

| | | Surface tension |
|---|---|---|
| | | reducing compound |
| | | (prepared from yerba |
| | | mate with ~50% |
| Sample | Steviol glycoside | dicaffeoylquinic acids) |
| 1 | 500 ppm RebA (~95%) | — |
| 2 | 500 ppm RebA (~95%) | 500 ppm |
| 3 | 500 ppm RebD (>99%) | — |
| 4 | 500 ppm RebD (>99%) | 500 ppm |
| 5 | 500 ppm RebM (>99%) | — |
| 6 | 500 ppm RebM (>99%) | 500 ppm |
| 7 | 500 ppm RA80 | — |
| 8 | 500 ppm RA80 | 500 ppm |
| 9 | — | — |
| 10 | — | 500 ppm |

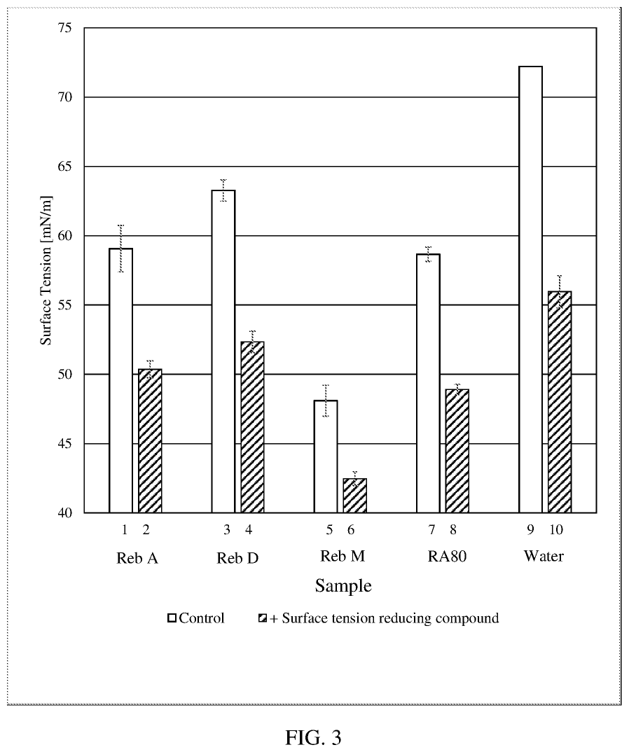

Surface tension measurements of each solution were determined by a Kruss K100MK2 Tensiometer with a roughened platinum and iridium Wilhelmy plate (19.9 mm width×0.2mm thickness×10 mm height) in a 45 mm diameter glass vessel at 20° C. The settings were as follows: detection speed, 10 mm/min; detection sensitivity, 0.005 g; immersion depth 2.0 mm, values, 10 (per log10 time decade); acquisition, logarithmic; maximum measuring time 21600 s; and vessel, SV10 glass vessel, 43.5 ml, 50 mm. The probe was cleaned with water and flame dried after each measurement. The glass vessel was cleaned thoroughly with water between samples. Deionized water was used as a reference between samples to ensure that no cross-contamination between plate and vessel occurred. Sample containers were gently inverted multiple times to promote thorough mixing. For each sample measurement, 40-50 ml of each sample was transferred to the glass vessel and immediately placed onto the sample stage. Each sample aliquot was magnetically stirred until the surface tension measurement was initiated to prevent surface aging. Samples were measured at equilibrium at 6 h and comprised an average of three data points across all replicates excluding outliers. The surface tension measurements are shown below in Table 3 and in FIG. 3.

TABLE 3

| | | Surface tension | Average | | |
|---|---|---|---|---|---|
| | | reducing compound | surface tension | | |
| | | (prepared from yerba | measurement | | Reduction |
| | Steviol | mate with ~50% | (mN/m at 20° | Standard | in surface |
| Sample | glycoside | dicaffeoylquinic acids) | at equilibrium) | deviation | tension |
| 1 | 500 ppm | — | 59.06623 | 1.67811 | |
| | RebA | | | | |
| | (~95%) | | | | |
| 2 | 500 ppm | 500 ppm | 50.35869 | 1.198573 | 14.74% |
| | RebA | | | | |
| | (~95%) | | | | |
| 3 | 500 ppm | — | 63.2594 | 0.765295 | |
| | RebD | | | | |
| | (>99%) | | | | |
| 4 | 500 ppm | 500 ppm | 52.33913 | 0.773653 | 17.26% |
| | RebD | | | | |
| | (>99%) | | | | |
| 5 | 500 ppm | — | 48.09162 | 1.112912 | |
| | RebM | | | | |
| | (>99%) | | | | |
| 6 | 500 ppm | 500 ppm | 42.46702 | 0.498555 | 11.70% |
| | RebM | | | | |
| | (>99%) | | | | |
| 7 | 500 ppm | — | 58.65504 | 0.519567 | |
| | RA80 | | | | |
| 8 | 500 ppm | 500 ppm | 48.90965 | 0.380981 | 16.61% |
| | RA80 | | | | |
| 9 | — | — | 72.2 | — | |
| 10 | — | 500 ppm | 55.96052 | 1.15176 | 22.49% |

The surface tension measurements showed that the surface tension was reduced for the rebaudioside A with surface tension reducing compound (Sample 2) compared to the rebaudioside A without surface tension reducing compound (Sample 1). The surface tension was reduced by about 14.74%. The surface tension measurements showed that the surface tension was reduced for the rebaudioside D with surface tension reducing compound (Sample 4) compared to the rebaudioside D without surface tension reducing compound (Sample 3). The surface tension was reduced by about 17.26%. The surface tension measurements showed that the surface tension was reduced for the rebaudioside M with surface tension reducing compound (Sample 6) compared to the rebaudioside M without surface tension reducing compound (Sample 5). The surface tension was reduced by about 11.70%. The surface tension measurements showed that the surface tension was reduced for RA80 with surface tension reducing compound (Sample 8) compared to RA80 without surface tension reducing compound (Sample 7). The surface tension was reduced by about 16.61%. The surface tension measurements showed that the surface tension was reduced for water with surface tension reducing compound (Sample 10) compared to water without surface tension reducing compound (Sample 9). The surface tension was reduced by about 22.49%. The surface tension measurements showed that the surface tension reducing compound reduced surface tension for each of the tested steviol glycoside systems.

### Example 3

A series of surface tension assays were carried out to characterize steviol glycoside composition with and without different surface tension reducing compounds. A steviol glycoside blend comprising primarily rebaudioside M (RM80) was used. Cichoric acid and rosmarinic acid were used as surface tension reducing compounds.

A control solution of water was prepared. A control solution of 500 ppm of cichoric acid surface tension reducing compound was prepared. A test solution of 500 ppm of RM80 blend and 500 ppm of cichoric acid surface tension reducing compound was prepared. A control solution of 500 ppm of rosmarinic acid surface tension reducing compound was prepared. A test solution of 500 ppm of RM80 and 500 ppm of rosmarinic acid surface tension reducing compound was prepared.

Surface tension measurements of each of the control solutions and test solutions were determined by a Kruss K100MK2 Tensiometer with a roughened platinum and iridium Wilhelmy plate in a 45 mm diameter glass vessel at 20° C. as described above. The surface tension of the control solution of water was 72.2 mN/m. The surface tension of the solution of 500 ppm of cichoric acid surface tension reducing compound was 71.67 mN/m. The surface tension of the test solution of 500 ppm of RM80 blend and 500 ppm of cichoric acid surface tension reducing compound was 45.46 mN/m. The surface tension of the control solution of 500 ppm of rosmarinic acid surface tension reducing compound was 63.08 mN/m. The surface tension of the test solution of 500 ppm of RM80 and 500 ppm of rosmarinic acid surface tension reducing compound was 45.6 nM/m.

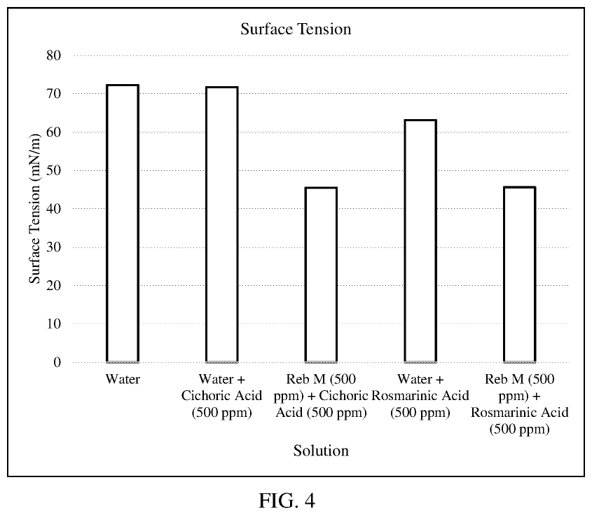

The surface tension measurements are shown below in Table 4 and in FIG. 4.

TABLE 4

| | | | Average |
|---|---|---|---|
| | | | surface tension |
| | Steviol | Surface tension | (mN/m at 20° at |
| Sample | glycoside | reducing compound | equilibrium) |
| Water | — | — | 72.2 |
| Cichoric | — | 500 ppm of cichoric | 71.67 |
| Acid | | acid | |
| RM80 + | 500 ppm of | 500 ppm of cichoric | 45.46 |
| cichoric | RM80 | acid | |
| acid | | | |
| Rosmarinic | — | 500 ppm of rosmarinic | 63.08 |
| acid | | acid | |
| RM80 + | 500 ppm of | 500 ppm of rosmarinic | 45.6 |
| rosmarinic | RM80 | acid | |
| acid | | | |

The surface tension of the control cichoric acid surface tension reducing compound was about the same as the water control, showing that the cichoric acid surface tension reducing compound did not significantly reduce the surface tension. The surface tension of the test solution of 500 ppm of RM80 blend and 500 ppm of cichoric acid surface tension reducing compound was reduced (45.46 mN/m) from the water control (72.2 mN/m) and the control cichoric acid surface tension reducing compound (45.46 mN/m), showing that the test solution had a reduced surface tension compared to the control solutions. The surface tension of the control rosmarinic acid surface tension reducing compound (63.08 mN/m) was reduced compared to the water control (72.2 mN/m), showing that the rosmarinic acid surface tension reducing compound did reduce the surface tension. The surface tension of the test solution of 500 ppm of RM80 blend and 500 ppm of rosmarinic acid surface tension reducing compound was reduced (45.6 mN/m) from the water control (72.2 mN/m) and the control rosmarinic acid surface tension reducing compound (63.08 mN/m), showing that the test solution had a reduced surface tension compared to the control solutions.

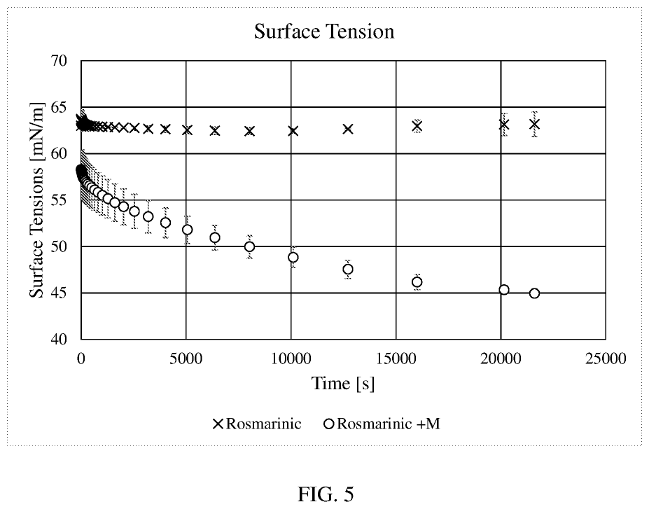

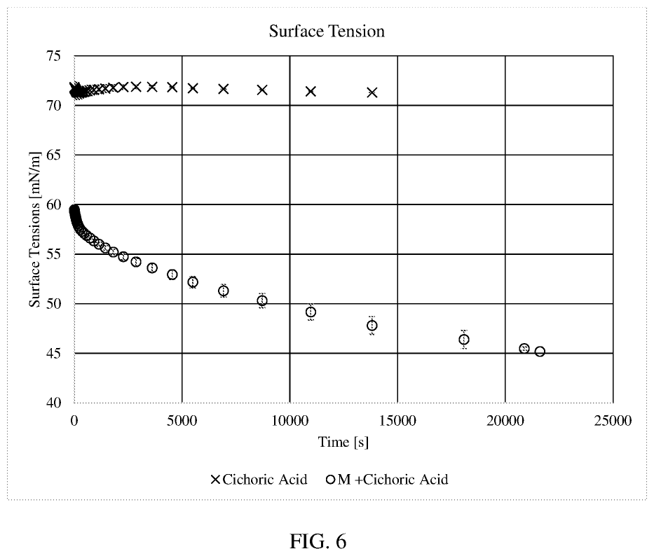

Kinetic surface plots of surface tension over time were also recorded for the control solution of 500 ppm of the rosmarinic acid surface tension reducing compound and the test solution of 500 ppm of RM80 and 500 ppm of rosmarinic surface tension reducing compound. FIG. 5 shows the kinetic surface plot of surface tension over time for the control solution of 500 ppm of the rosmarinic acid and the test solution of 500 ppm of RM80 and 500 ppm of rosmarinic surface tension reducing compound. FIG. 6 shows the kinetic surface plot of surface tension over time for the control solution of 500 ppm of the cichoric acid and the test solution of 500 ppm of RM80 and 500 ppm of cichoric acid surface tension reducing compound.

1.-50. (canceled)

51. A method for reducing surface tension in a steviol glycoside solution, the method comprising:
adding one or more steviol glycosides and a surface tension reducing compound to an aqueous solution to form a steviol glycoside solution; wherein surface tension of the steviol glycoside solution is reduced relative to an equivalent solution lacking the surface tension reducing compound;
wherein the surface tension reducing compound comprises:
one or more monocaffeoylquinic acids or salts thereof;
(ii) at least 20% (wt) of one or more dicaffeoylquinic acids or salts thereof; and
(iii) at least one of an ester of ferulic acid and quinic acid, an ester of sinapic acid and quinic acid, an ester of p-coumaric acid and quinic acid, an ester of caffeic acid and 3-(3,4-dihydroxyphenyl)lactic acid, an ester of caffeic acid and tartaric acid, and isomers thereof; and
wherein the one or more steviol glycosides and the surface tension reducing compound are added such that the steviol glycoside solution comprises a 1:0.5 to 1:4 ratio by weight of steviol glycose to surface tension reducing compound.

52. The method of claim 51, wherein surface tension is reduced by at least 10%, at least 20%, at least 30%, at least 40%, at least 50%, at least 60%, at least 70%, at least 80% or at least 90% compared to a steviol glycoside solution without the surface tension reducing compound.

53. The method of claim 51, wherein the surface tension reducing compound comprises at least 30% (wt) of a total of 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, 4,5-dicaffeoylquinic acid, and salts thereof.

54. The method of claim 51, wherein the one or more monocaffeoylquinic acids or salts thereof comprise one or more of neochlorogenic acid, cryptochlorogenic acid, 1,3-dicaffeoylquinic acid, and salts thereof.

55. The method of claim 51, wherein the surface tension reducing compound comprises one or more of 3-O-feruloylquinic acid, 4-O-feruloylquinic acid, 5-O-feruloylquinic acid, 3,4-diferuloylquinic acid, 3,5-diferuloylquinic acid, 4,5-diferuloylquinic acid, and salts thereof.

56. The method of claim 51, wherein the steviol glycoside solution comprises between about 100 ppm and about 5000 ppm steviol glycoside.

57. The method of claim 51, wherein the one or more steviol glycosides comprises rebaudioside D, rebaudioside M, or combination thereof.

58. The method of claim 51, wherein the steviol glycoside solution comprises between 50 wt % and 95 wt % rebaudioside M based on total weight of steviol glycosides in the solution.

59. The method of claim 51, wherein the steviol glycoside solution comprises between 5 wt % and 25 wt % rebaudioside D based on total weight of steviol glycosides in the solution.

60. The method of claim 51, wherein the steviol glycoside solution comprises at least 1 wt % steviol glycosides.

61. The method of claim 51, wherein the steviol glycoside solution comprises a 1:1 to 1:3 ration by weight of steviol glycoside to surface tension reducing compound.

62. The method of claim 51, wherein the aqueous solution comprises a 1:1 to 1:3 ratio by weight of steviol glycoside to total concentration of esters of caffeic acid and quinic acid, esters of ferulic acid and quinic acid, esters of sinapic acid and quinic acid, esters of p-coumaric acid and quinic acid, esters of caffeic acid and 3-(3,4-dihydroxyphenyl)lactic acid, and esters of caffeic acid and tartaric acid.

63. The method of claim 51, additionally comprising the step of preparing a beverage comprising the steviol glycoside solution.

64. A method for preparing a beverage concentrate solution with reduced surface tension, the method comprising:
adding to an aqueous solution i) one or more steviol glycosides and ii) a surface tension reducing compound to form a beverage concentrate solution, wherein the beverage concentrate solution has reduced surface tension relative to an equivalent beverage concentrate solution lacking the surface tension reducing compound;
wherein the surface tension reducing compound comprises:
one or more monocaffeoylquinic acids or salts thereof;
(ii) at least 20% (wt) of one or more dicaffeoylquinic acids or salts thereof; and
(iii) at least one of an ester of ferulic acid and quinic acid, an ester of sinapic acid and quinic acid, an ester of p-coumaric acid and quinic acid, an ester of caffeic acid and 3-(3,4-dihydroxyphenyl)lactic acid, an ester of caffeic acid and tartaric acid, and isomers thereof; and
wherein the one or more steviol glycosides and the surface tension reducing compound are added such that the beverage concentrate solution comprises a 1:0.5 to 1:4 ratio by weight of steviol glycose to surface tension reducing compound.

65. The method of claim 64, wherein the beverage concentrate solution comprises 2400 to 4200 ppm of total steviol glycoside.

66. The method of claim 64, wherein the beverage concentrate solution comprises 1800 to 5400 ppm surface tension reducing compound.

67. The method of claim 64, wherein the beverage concentrate solution includes a buffer and has a pH between 1.5 and 4.

68. The method of claim 64, wherein surface tension is reduced by at least 10%, at least 20%, at least 30%, at least 40%, at least 50%, at least 60%, at least 70%, at least 80% or at least 90% compared to a steviol glycoside solution without the surface tension reducing compound.

69. The method of claim 64, wherein the surface tension reducing compound comprises at least 30% (wt) of a total of 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, 4,5-dicaffeoylquinic acid, and salts thereof.

70. The method of claim 64, wherein the one or more steviol glycosides comprises rebaudioside D, rebaudioside M, or combination thereof.

71. The method of claim 64, wherein the steviol glycoside solution comprises between 50 wt % and 95 wt % rebaudioside M and 5 wt % and 25 wt % rebaudioside D based on total weight of steviol glycosides in the solution.

72. The method of claim 64, wherein the steviol glycoside solution comprises at least 1 wt % steviol glycosides.

73. The method of claim 64, wherein the aqueous solution comprises a 1:1 to 1:3 ratio by weight of steviol glycoside to total concentration of esters of caffeic acid and quinic acid, esters of ferulic acid and quinic acid, esters of sinapic acid and quinic acid, esters of p-coumaric acid and quinic acid, esters of caffeic acid and 3-(3,4-dihydroxyphenyl)lactic acid, and esters of caffeic acid and tartaric acid.
